(19) United States
(12) Patent Application Publication
(10) Pub. No.: US 2016/0235677 A1
(43) Pub. Date: Aug. 18, 2016

(54) METHOD OF CONVERTING A CRYSTALLINE COMPOUND TO AN AMORPHOUS COMPOUND, METHOD OF INCREASING THE SOLUBILITY OF A CRYSTALLINE COMPOUND IN A BIORELEVANT FLUID, AND NANOPARTICLES THAT ACHIEVE SUPERSATURATION

(71) Applicant: Nanocopoeia, LLC., St. Paul, MN (US)

(72) Inventors: Robert A. Hoerr, Shoreview, MN (US); Huijing Fu, Arden Hills, MN (US); James E. Lasch, Oakdale, MN (US); Mourad F. Rahi, Maple Grove, MN (US)

(21) Appl. No.: 14/952,771

(22) Filed: Nov. 25, 2015

Related U.S. Application Data
(60) Provisional application No. 62/084,277, filed on Nov. 25, 2014.
(60) Provisional application No. 62/084,291, filed on Nov. 25, 2014.

(51) Int. Cl. A61K 9/16; A61J 3/02; A61K 31/573; A61K 31/407; A61K 31/58; B05B 5/025; A61K 31/404; A61K 31/635; A61K 31/5415; A61K 31/196; A61K 31/5377; A61K 31/496; A61K 31/192

(52) U.S. Cl. CPC A61K 9/1694; A61K 9/1635; A61K 31/496; A61J 3/02; A61K 31/573; A61K 9/1641; A61K 31/407; A61K 31/58; A61K 31/192; A61K 31/404; A61K 9/1652; A61K 31/635; A61K 31/5415; A61K 31/196; A61K 31/5377; B05B 5/025

(57) ABSTRACT

A method of converting a poorly water soluble crystalline compound to an amorphous compound and a method of increasing the solubility of a poorly water soluble crystalline compound in biorelevant fluid at pH 6.5 is disclosed. The method includes dissolving the compound and a polymer in a solvent to form a solution, the polymer being present in the solution in an amount such that after electrospraying the solution the compound is in an amorphous form, electrospraying the solution using an electrospray system, the electrospraying forming nanoparticles, collecting the nanoparticles on a substrate, and removing the nanoparticles in the form of a powder.

### CROSS REFERENCE TO RELATED APPLICATIONS

This application claims the benefit of U.S. Provisional Application No. 62/084,277, filed Nov. 25, 2014, and U.S. Provisional Application No. 62/084,291, filed Nov. 25, 2014, both of which are incorporated herein.

### BACKGROUND

The invention is directed to improving the solubility of poorly water soluble crystalline compounds and increasing throughput of electrospray systems.

Drug manufacturing is a complex process that often presents significant challenges during the various stages of product formulation. Formulating poorly water soluble drugs that exhibit enhanced bioavailability and consistent drug delivery is particularly challenging. Various formulation techniques have been used to increase drug dissolution. These techniques focused primarily on particle size reduction and conversion of the crystalline form to amorphous with commonly used polymers and excipients. Examples of formulation techniques include hot melt extrusion, solid dispersion, spray drying, micronization by flash evaporation, ultra-rapid freezing, self-emulsifying drug delivery system, liposomal dispersion using freeze-drying, mixed polymeric micelle formulation, electrospinning of nanofibers, controlled precipitation, evaporative precipitation, cocrystals formation, high sheer mixing, nanosized crystals prepared via wet milling, mucoadhesive in situ gel formulation, solid dispersions including aerosol solvent extraction with supercritical fluid, kneading, co-evaporation, microwave irradiation, freeze dry followed by lyophilization, rotary evaporator drying, and flocculation.

Various investigations have concluded that drug formulations leading to prolonged supersaturation enhance drug absorption.

BCS Class II drugs exhibit poor solubility in both aqueous and commonly used organic solvents. Approaches for improving the solubility of BCS Class II drugs fall into two main categories: reducing particle size to increase surface area or converting from a crystalline to amorphous form. Both approaches employ polymers and surfactants to prevent the product from reverting to the more stable, but less soluble crystalline state. Innovations for improving solubility and achieving commercial-scale use include wet milling, which produces drug nanocrystals, heat melt extrusion, and solvent spray drying which yield solid dispersions that stabilize the drug in its amorphous form. Milling is a “top-down” particle-forming process, where large drug crystals and excipient are reduced to smaller particles and stabilized in crystalline or semi-disordered crystals with the excipient. Hot melt extrusion forms liquid dispersions at high temperatures that are milled into particles after cooling. Solvent-spray drying is a “bottom-up” process where the particles are formed directly from solution. Both solvent-spray drying and hot melt extrusion have been used to form solid dispersions of poorly water soluble drugs with polymers such as hydroxypropyl methycellulose-acetate succinate (HPMCAS), polyvinyl caprolactam-polyvinyl acetate-polyethylene glycol graft copolymer (SOLUPLUS, or SP), amino methacrylate copolymer-NF (EUDRAGIT E100 or EPO), methacrylic acid copolymer type C-NF (EUDRAGIT L100-55, and carbomer homopolymer type B-NF (CARBOPOL 974P). Milling, solvent-spray drying, and hot melt extrusion each operate at high temperatures or require heat.

Itraconazole and griseofulvin are two examples of antifungal drugs that fall within this class of drugs. Griseofulvin (GF) is a BCS Class II crystalline drug with poor water solubility, reported to be 8.64 mg/L at 25° C. Itraconazole (ITZ) is an antifungal agent used for the treatment of local and systemic fungal infections. Itraconazole is also categorized as a BCS Class II drug due to its poor solubility in both aqueous and commonly used organic solvents, characterized by low solubility, a pKₐ=3.7, and high permeability (log P=6.2). Three commercial formulations of ITZ products are available under the commercial brand SPORANOX, including oral capsules, oral solution, and intravenous infusion formulations. SPORANOX capsules are prepared by solid dispersion of ITZ with sugar beads coated with hydroxypropylmethylcellulose and polyethylene glycol. The commercially available oral and intravenous solutions are formulated in a 1:40 ratio by weight with hydroxypropyl-β-cyclodextrin, a solubilizing and complexing agent that has been associated with rare cases of serious hepatotoxicity, including liver failure.

The reported aqueous solubility of itraconazole varies substantially from 1 ng/mL in neutral pH to 4 μg/mL in acidic media. Since its oral solubility is pH- and food-dependent, it has unpredictable bioavailability, and is reported to reach a maximum absorption of 55% when it is taken immediately after a full meal or with an acidic beverage. The absorption of itraconazole in the stomach is minimal compared to the large absorptive surface area of the intestinal mucosa and its lipophilic properties require an oral sustained-release formulation to ensure passage of the drug through low pH environment of the stomach and delivery into the small intestine.

Most of the itraconazole solubilization methods reported in the literature focused on drug release in acidic media. These studies report good dissolution results at low pH, but no significant improvement from dissolution under simulated intestine fluid in fasted state (FaSSIF) conditions over SPORANOX.

Efforts have been made to formulate itraconazole with excipients capable of maintaining a slow and sustained release of the drug but their results have fallen short of expectations as the concentration of itraconazole dropped rapidly following acid-to-neutral transition. Further investigations conducted showed that the addition of carbomer homopolymer (CARBOPOL 974P) to the melt extruded mixtures provided improvement in the duration of supersaturation and enhanced bioavailability compared to SPORANOX pellets.

Electrospray, which has also been referred to as electrohydrodynamic atomization, has been used to produce nanoparticles from solutions and colloidal suspensions. Cone-jet electrospray, first described more than two decades ago, emits a plume of unipolar charged micro- or nano-scale droplets at a critical high voltage. The voltage required to form the cone-jet depends on the system geometry and the conductivity and composition of the feed liquid. Once formed, the cone-jet mode is stable, without corona discharge, provided that the high voltage power is carefully regulated and the flow of feed liquid is held constant. The emitted plume is composed of microdroplets of solvent and dissolved solids, which carry a unipolar surface charge that repels adjacent droplets. During the brief flight path, the solvent flashes off from the droplets, resulting in dry, uniform particles. Unfortunately, the small throughput of a single capillary nozzle has limited the use of electrospray to laboratory scale demonstrations.

There is a need for an improved oral formulation with greater bioavailability that could improve the safety and efficacy of itraconazole.

There is a need to enhance the bioavailability of drugs that exhibit low water solubility, such as BCS class II drugs, a need to improve the solubility of low water soluble drugs, and a need to find approaches to improving drug solubility that do not require relatively high temperatures.

### SUMMARY

In one aspect, the invention features a method of making a poorly water soluble crystalline compound an amorphous compound, the method including dissolving the crystalline compound and a polymer in a solvent to form a solution, electrospraying the solution using an electrospray device, the electrospraying forming amorphous nanoparticles, and collecting the nanoparticles, the nanoparticles including the compound in an amorphous form.

In another aspect, the invention features a method of increasing the solubility of a poorly water soluble crystalline compound in a biorelevant fluid having a pH of at least 6.5, the method including dissolving the compound and a polymer in a solvent to form a solution, the polymer being present in the solution in an amount such that, after electrospraying the solution, the compound is in an amorphous form, electrospraying the solution using an electrospray device, the electrospraying forming nanoparticles, and collecting the nanoparticles, the nanoparticles include the compound in an amorphous form.

In some embodiments, the method further includes collecting the nanoparticles on a substrate, and removing the nanoparticles from the substrate to form a powder of the nanoparticles. In some embodiments, the polymer is present in the solution in an amount such that, after electrospraying the solution, the compound is in an amorphous form.

In one embodiment, the method further includes dissolving the compound in a first solvent to form a first solution, dissolving the second compound in a second solvent to form a second solution, and combining the first solution and the second solution to form a third solution, the electrospraying including electrospraying the third solution using the electrospray device.

In some embodiments, the nanoparticles are amorphous. In other embodiments, the nanoparticles are amorphous and remain amorphous when stored in a sealed container for at least one week at room temperature. In another embodiment, the nanoparticles are amorphous and remain amorphous when stored in a sealed container for at least two weeks at room temperature.

In one embodiment, the nanoparticles exhibit a smaller particle size in FaSSIF having a pH of at least 6.5 relative to the particle size of the compound, electrosprayed in the absence of the polymer, in FaSSIF having a pH of at least 6.5.

In other embodiments, the nanoparticles achieve supersaturation of the amorphous compound in FaSSIF having a pH of at least 6.5. In some embodiments, the nanoparticles achieve supersaturation of the amorphous compound in FaSSIF having a pH of at least 6.5 for a period of at least 60 minutes. In other embodiments, the nanoparticles achieve supersaturation of the amorphous compound in FaSSIF having a pH of at least 6.5 for a period of at least 60 minutes, after exposure to FaSSGF having a pH of 1.6 for 30 minutes. In another embodiment, the nanoparticles achieve supersaturation of the amorphous compound in FaSSIF having a pH of at least 6.5 for a period of at least 120 minutes. In one embodiment, the nanoparticles achieve supersaturation of the amorphous compound in FaSSGF having a pH of no greater than 1.6.

In some embodiments, the nanoparticles exhibit a smaller particle size in FaSSGF having a pH of no greater than 1.6 relative to the size of particles of the compound electrosprayed in the absence of polymer in FaSSGF having a pH of no greater than 1.6. In other embodiments, the nanoparticles achieve a greater solubility of the amorphous compound in FaSSIF having a pH of at least 6.5 than in FaSSGF having a pH no greater than 1.6. In another embodiment, the nanoparticles achieve a greater solubility of the amorphous compound in FaSSGF having a pH no greater than 1.6 than in FaSSIF having a pH of at least 6.5.

In some embodiments, the nanoparticles exhibit no greater than one phase transition.

In some embodiments, the polymer is an amorphous, water insoluble polymer. In one embodiment, the polymer is a cationic polymer. In another embodiment, the compound is anionic and the polymer is a cationic polymer. In another embodiment, the polymer comprises an anionic polymer and at least one proton donating group.

In one embodiment, the crystalline compound is a crystalline drug. In some embodiments, the crystalline compound is at least one of an antifungal drug, a non-steroidal anti-inflammatory drug, a corticosteroid, and a substance P antagonist. In one embodiment, the compound is a crystalline drug. In some embodiments, the compound is itraconazole. In other embodiments, the compound is griseofulvin.

In another embodiment, the method further includes removing the nanoparticles from the substrate such that the nanoparticles are in the form of a powder.

In another aspect, the invention features a particulate that includes amorphous nanoparticles that include an amorphous compound and an amorphous polymer, the nanoparticles having been formed by electrospraying a solution of the compound, the polymer, and solvent from an electrospray device. In one embodiment, the particulate is a free flowing powder. In other embodiments, the nanoparticles include spheroidal nanoparticles with surface dimpling, discoid nanoparticles, teardrop-spheroidal nanoparticles, wrinkled spheroidal nanoparticles, porous spheroidal nanoparticles, pitted spheroidal nanoparticles, or a combination thereof.

In another embodiment, the method further includes adding a sufficient amount of polymer to the solvent such that the nanoparticles exhibit no greater than one phase transition.

In other embodiments, the method further includes adding a sufficient amount of polymer to the solvent such that the nanoparticles are amorphous.

In other aspects, the invention features a method of formulating a drug, the method including dissolving a poorly water soluble crystalline drug and a polymer in a solvent to form a solution, the polymer being present in the solution in an amount such that after electrospraying the solution the compound is in an amorphous form, electrospraying the solution using an electrospray device to form nanoparticles, and collecting the nanoparticles created by the electrospraying on a substrate.

In another aspect, the invention features a method of making amorphous itraconazole from crystalline itraconazole, the method including dissolving crystalline itraconazole and a polymer in a solvent to form a solution, electrospraying the solution using an electrospray device, the electrospraying forming nanoparticles, and collecting the nanoparticles on a substrate.

In other aspects, the invention features an electrospray system including a plurality of nozzles that include a source end, a spray end, a first component that includes a first surface, a second component that includes a second surface, a fluid channel defined between the first surface and the second surface, the fluid channel forming an exit slit at the spray end of the nozzle, and a plurality of electrically chargeable notches located on at least one of the first component and the second component proximate the exit slit, a voltage source electrically coupled to at least one of the nozzle and a target substrate and configured to establish an electric field between the target substrate and the spray end of the nozzle, and a syringe pump in flow communication with the source end of the nozzle, the syringe pump configured to propel a spray liquid through the nozzle.

In one embodiment, the nozzles are positioned in a linear array. In other embodiments, the nozzles are positioned in a circular array. In another embodiment, the nozzles are positioned multiple linear arrays. In some embodiments, the nozzles are positioned multiple linear arrays where the nozzles of a first linear array are offset from the nozzles of a second linear array.

The invention features an electrospray system that includes arrays of multi jet nozzles. The invention also features an electrospray process that produces powdered formulations of drug and polymer, that can be performed at ambient conditions, that can generate powders of submicron particles in a single-step process, that allows rapid quenching of sprayed particles, and that yields powders of submicron particles in an amorphous form. The multi-nozzle electrospray process provides a relatively high throughput.

The invention also features an electrospray process that is capable of producing high drug loading in solid dispersions.

The invention also features a process for formulating drugs that exhibit low water solubility in a single-step electrospray process that can be performed at ambient temperature and pressure, and that can enable the rapid conversion of a compound from crystalline form to amorphous form using a variety of readily available excipients. The electrospray process is well suited for use in conjunction with thermally labile compounds.

The invention also features drug formulations that include drug in an amorphous form and that exhibit good stability and good dissolution profiles.

Other features and advantages will be apparent from the following brief description of the drawings, the drawings, the description of the preferred embodiments, and from the claims.

### GLOSSARY

In reference to the invention, these terms have the meanings set forth below:

The term “poorly water soluble crystalline compound” means a crystalline compound that is slightly soluble, very slightly soluble, or practically insoluble in water.

The term “slightly soluble in water” means from 100 parts to 1000 parts water are required to solubilize one part solute.

The term “very slightly soluble in water” means from 1000 parts to 10,000 parts water are required to solubilize one part of solute.

The term “practically insoluble in water” means at greater than 10,000 parts water are required to solubilize one part of solute.

The term “supersaturation” means a concentration of a solute in a solvent that is greater than the equilibrium solubility that is achieved by the crystalline form of the solute in that solvent.

The term “drug” means a substance that has a physiological effect when introduced into the body.

The term “electrospraying” means spraying a fluid that includes an organic solvent through a nozzle toward a substrate in the presence of a non-uniform electric field that exists between the nozzle and the substrate, such that the fluid forms a cone jet at the spray end of the nozzle and disperses into fine droplets as a result of the electric field and the droplets accelerate toward the substrate as a result of the non-uniform electric field.

### BRIEF DESCRIPTION OF THE DRAWINGS

FIG. 1 is a diagram of one embodiment of a high throughput, multi-nozzle, multi-jet electrospray system that includes two linear arrays of nozzles in an offset relationship.

FIG. 2A is a photograph of D12 and D24 nozzles, which have a circular form and boundary of inner notched ring and outer tapered cylinder. The spray solution is fed from the top of the nozzle and flows as a sheet between the inner and outer cylinders, terminating at the edge of the notched ring.

FIG. 2B is a photograph of the bottom end of a nozzle that includes 24 notches on the inner ring of the nozzle.

FIG. 3 is a photograph of the D24 nozzle of FIG. 2B spraying in cone-jet mode showing the 24 independent plumes emitting from the notched ring taken from the side of the nozzle.

FIGS. 4A-4D are four scanning electron microscope (SEM) images of griseofulvin (GF) formulation particles produced by a D12 nozzle.

FIGS. 5A-5F are six SEM images of GF formulation particles produced by a D24 nozzle.

FIGS. 6A and 6B are plots of particle size in liquid after powders dissolved in fasted-state simulated intestinal fluid medium (FaSSIF) as measured by nanoparticle tracking analysis.

FIGS. 7A and 7B are bar graphs showing the results of materials tested in the Caco-2 cell monolayers, which materials included griseofulvin powder, as received, and three ENS-processed formulations: GF:SP 1:1, GF:SP:SDS 1:1:0.1, and GF:SP:DS 1:1:0.1. For each of the formulations, the value (mean±SD) obtained for the initial apical side GF concentration (left) (FIG. 7A) and flux (right) (FIG. 7B) is compared to that for the GF control (*p<0.05, **p<0.01, †p<0.005). Flux was calculated from the griseofulvin concentration in the basolateral side fluid at 2 hours.

FIGS. 8A and 8B are bar graphs showing the effect of griseofulvin dose level on Caco-2 cell flux. The materials tested included griseofulvin powder, GF-ENS, GF:SP-1:1-physical mixture (PM), GF:SP-1:1-solvent evaporation (SE), and GF:SP-1:1-ENS. For each cell monolayer, the initial apical side GF concentration in μg/mL is reported for a GF dose of 200 μg or 500 μg. The corresponding flux in μg/sec was calculated from GF concentration in the basolateral side fluid at 2 hours. Each value plotted is the mean±SD replicate measurements.

FIG. 9 are plots of dissolution profiles for GF powder, GF:SP physical mixtures, and GF ENS powders formed with a D24-nozzle. The graph shows results for three unprocessed powders (GF, GF:SP 1:2 and 1:5 physical mixtures) and five ENS-processed powders (GF, GF:SP 1:0.5, 1:1, 1:2 and 1:5).

FIGS. 10A and 10B are differential scanning calorimetry (DSC) thermograms. The top thermogram of GF:SP-1:1 ENS was obtained with a slow heating rate of 1° C./min, used to increase resolution, as shown in FIG. 10A. The tracing shows three large peaks corresponding to residual solvent evaporation, partial recrystallization of amorphous griseofulvin while heating in the DSC and melting of the resulting crystalline griseofulvin. The bottom tracing compares GF:SP-1:1-ENS to GF:SP-PM and unprocessed GF powder, as shown in FIG. 10B. The ENS-processed material shows a peak for the negative heat flow of crystallization.

FIG. 11 shows the X-ray diffraction spectra for griseofulvin powder, SOLUPLUS powder, and ENS-processed griseofulvin and GF:SP-1:0.5, -1:1, -1:2 and -1:5, respectively.

FIG. 12 shows the Fourier transform infrared spectroscopy absorbance spectra for the following materials: GF powder, as received; GF:SP 1:1 physical mixture; GF:SP 1:1 ENS processed; and SP powder, as received. The spectra are overlaid for comparison. Reference wave numbers showing peak differences among the samples are marked by dotted vertical lines and labels.

FIG. 13A is a scanning electron microscopy image, on a 10 μm scale, of a sprayed formulation of itraconazole (ITZ):EUDRAGIT L100-55 at a ratio of 1:2.

FIG. 13B is a scanning electron microscopy image, on a 10 μm scale, of a sprayed formulation of ITZ:EUDRAGIT E100 at a ratio of 1:2.

FIG. 13C is a scanning electron microscopy image, on a 10 μm scale, of a sprayed formulation of ITZ.

FIG. 13D is a scanning electron microscopy image, on a 10 μm scale, of a sprayed formulation of 1% K30 polyvinylpyrrolidone.

FIGS. 14A and 14B are differential scanning calorimetry (DSC) analyses of ITZ and EUDRAGIT L100-55 neat, as physical mixtures and as formed using ENS at various ITZ:EUDRAGIT L100-55 ratios where FIG. 14A is a heat flow DSC and FIG. 14B is a reversed heat flow DSC.

FIGS. 14C and 14D are DSC analyses of ITZ and EUDRAGIT E100 neat, as physical mixtures, and as formed using ENS at various ITZ:EUDRAGIT E100 ratios where FIG. 14C is a heat flow DSC and FIG. 14D is a reversed heat flow DSC.

FIG. 15 is a Fourier transform infrared scans showing the infrared absorption of EUDRAGIT L100-55, itraconazole, a physical mixture of ITZ and EUDRAGIT L100-55 at a ratio of 1:1, ENS powder of ITZ and EUDRAGIT L100-55 at a ratio of 1:1, ITZ protonated with HCl, and subtracted profile of ENS-EUDRAGIT L100-55.

FIG. 16 includes structures that show the potential hydrogen bonding sites between itraconazole and EUDRAGIT L100-55.

FIG. 17A includes plots of non-sink dissolution testing in FaSSIF medium having a pH of 6.5. Two levels of electrosprayed powder of itraconazole:EUDRAGIT L100-55 at a 1:1 ratio, one added at 10 μg/mL of medium and the other added at 6.6 μg/mL, were compared with a physical mixture of itraconazole:EUDRAGIT L100-55 at a 1:1 ratio, added at 5.5 μg/mL under the same conditions.

FIGS. 17B and 17C include plots of acidic-to-neutral transition in buffer media: FIG. 17B ENS itraconazole:EUDRAGIT L100-55 and FIG. 17C ENS itraconazole:EUDRAGIT E100. Each dissolution vessel (n=3) contained 80 μg/mL itraconazole equivalent of each ENS powder used. Testing was conducted for 2 hrs in 75 mL 0.1 N HCl at pH 1.0, the acidic phase buffer, followed by pH adjustment to 6.8±0.05 (the neutral phase buffer), by adding 25 mL 0.2 M tribasic sodium phosphate solution to the acidic buffer.

FIGS. 17D and 17E include plots of acidic-to-neutral transition in biorelevant media: FIG. 17D, ENS itraconazole:EUDRAGIT L100-55, and FIG. 17E, ENS itraconazole:EUDRAGIT E100. Each dissolution vessel (n=3) contained 80 μg/mL itraconazole equivalent of each ENS powder used. Testing was conducted for 2 hrs in 75 mL fasted state simulated gastric fluid (FaSSGF) pH 1.6. The pH transition to FaSSIF at pH 6.5±0.05 was accomplished with the addition of Transition Medium, as described in Dissolution in Biorelevant Media Test Method.

FIG. 18 are the dissolution profiles of Examples A7 and A8 and Controls A1 and A2.

FIG. 19A are the dissolution profiles of Example A8 and Control A2 as determined in a μDiss system.

FIG. 19B are the dissolution profiles of Example A7 and Control A2 as determined in a μDiss system.

FIG. 20 is a plot of particle size of ENS-sprayed itraconazole as determined by laser diffraction in biorelevant fluid over time and pH transition from pH 1.6 to 6.5

FIG. 21 is a plot of particle size of nanoparticles of ENS-sprayed itraconazole and EUDRAGIT L100-55 as determined by laser diffraction in biorelevant fluid over time and pH transition from pH 1.6 to 6.5.

FIG. 22 is a plot of particle size of nanoparticles of ENS-sprayed itraconazole and EUDRAGIT E100 as determined by laser diffraction in biorelevant fluid over time and pH transition from pH 1.6 to 6.5.

FIG. 23 are the XRD spectra of, from top to bottom, Control A1 and Examples A7 and A8.

FIG. 24A are plots of the percent dissolved aprepitant in FaSSIF versus time of (from bottom to top) Control C1 and Examples C2, C1, C3 and C4, respectively.

FIG. 24B are plots of the percent dissolved aprepitant in FaSSGF versus time of (from bottom to top) Control C1 and Examples C4, C3, C2, and C1.

### DETAILED DESCRIPTION

The method of converting a poorly water soluble crystalline compound to an amorphous compound and the method of increasing the solubility of a poorly water soluble crystalline compound (e.g., a poorly water soluble crystalline drug) in a biorelevant fluid at pH 6.5 include dissolving the compound and a polymer in a solvent to form a solution, electrospraying the solution using an electrospray system to form nanoparticles, and collecting the nanoparticles on a substrate, and optionally removing the nanoparticles from the substrate such that the nanoparticles are in the form of a powder (e.g., dry powder, dry flakes, non-agglomerated powder, and flowable powder (e.g., free flowing powder)). The resulting nanoparticles are amorphous and include an amorphous compound (e.g., a drug) and a polymer.

The method can be used to form amorphous compounds (e.g., amorphous drugs such as amorphous itraconazole and amorphous griseofulvin) from crystalline compounds (e.g., crystalline drugs such as crystalline itraconazole and crystalline griseofulvin).

One useful measure of the presence of an amorphous nanoparticle is differential scanning calorimetry (DSC). Preferably the nanoparticles exhibit no cystallinity when analyzed using DSC. Another useful method of determining whether a compound is in an amorphous form is X-Ray Powder Diffraction (XRD). Preferably the XRD spectrum of the amorphous nanoparticle is essentially free of or even free of individual sharp crystalline peaks and shows a halo characteristic of an amorphous material.

The amorphous nanoparticles also maintain the compound in an amorphous state for a period of at least 24 hours, at least 3 days, at least 7 days, at least 14 days, at least 5 weeks, at least 7 weeks, at least 10 weeks, at least 15 weeks, or even at least 20 weeks when stored in a sealed container at room temperature (i.e., from 20° C. to 25° C.) or even at room temperature and ambient humidity.

The amorphous nanoparticles also preferably exhibit a smaller particle size in a biorelevant fluid having a pH of 6.5 relative to the particle size exhibited by the electrosprayed crystalline compound in the same biorelevant fluid. Useful methods of measuring particle size in a biorelevant fluid include, e.g., particle size measurement in liquid using laser diffraction or nanoparticle tracking analysis test methods.

The amorphous nanoparticles preferably exhibit supersaturation in a biorelevant fluid (e.g., in a buffer media, in FaSSIF, FeSSIF, FaSSGF, and FeSSGF) for a period of at least 60 minutes, at least 120 minutes, at least 180 minutes, at least 240 minutes or even at least 300 minutes. The amorphous nanoparticles preferably exhibit supersaturation in a biorelevant fluid having a pH of at least 6.5 (e.g., in a buffer media having a pH of at least 6.5 or even in FaSSIF having a at least pH of 6.5) for a period of at least 60 minutes, at least 120 minutes, at least 180 minutes, at least 240 minutes or even at least 300 minutes, a biorelevant fluid having a pH of no greater 1.6 (e.g., in a buffer media having a pH of no greater than 1.6 or even in FaSSGF having a pH of no greater than 1.6) for a period of at least 60 minutes, at least 120 minutes, at least 180 minutes, at least 240 minutes or even at least 300 minutes, or a combination thereof.

The amorphous nanoparticles optionally exhibit sustained release of the amorphous compound, or even sustained release of the amorphous compound at supersaturated levels relative to the crystalline compound, in a biorelevant fluid having a pH of at least 6.5 or even a pH no greater than 1.6, preferably for a period of at least 120 minutes, at least 180 minutes, at least 240 minutes or even at least 300 minutes.

The amorphous nanoparticles also can be formulated to exhibit a greater solubility in a biorelevant fluid having a pH of at least 6.5 than in an acidic solution having a pH no greater than 1.6, or even a pH no greater than 1.5. Alternatively or in addition, the amorphous nanoparticles can be formulated to exhibit a greater solubility in a biorelevant fluid having a pH no greater than 1.6 than in a biorelevant fluid having a pH of at least 6.5.

The amorphous nanoparticles also preferably exhibit a dissolution profile in a biorelevant fluid having a pH of at least 6.5 or even a biorelevant fluid having a pH no greater than 1.6 such that the area under the curve is at least 35% greater, at least 40% or even at least 50% greater than the area under the curve of the dissolution profile of the crystalline compound in the biorelevant fluid. Useful methods for obtaining and analyzing the dissolution profile of the amorphous nanoparticles include the Dissolution in Biorelevant Media with pH Transition Test Method, the Dissolution in Biorelevant Buffer with pH Transition Test Method, and the Dissolution in Biorelevant Medium Using Non-Sink Condition Test Method.

The poorly water soluble crystalline compound and the polymer can be present in the nanoparticles in any suitable weight to weight (w/w) ratio including, e.g., at least about 1:0.5, at least about 1:1, at least about 1:2, at least about 1:4, at least about 1:5, at least about 1:6, at least about 1:7, at least about 1:8, or even at least about 1:10 (compound:polymer). Preferably the polymer is present in the spray composition and the resulting nanoparticles in an amount sufficient to maintain the compound in an amorphous form, or even in an amount sufficient to maintain the compound in an amorphous form for an extended period of time.

The greatest cross-sectional dimension of the nanoparticles (e.g., the diameter of a spherical nanoparticle) is no greater than 2000 nm, no greater than about 1000 nm, no greater than about 500 nanometers, no greater than about 100 nm, no greater than about 50 nm, from about 1 nm to about 2000 nm, from about 1 nm to about 500 nm, or even from about 1 nm to about 100 nm.

Useful nanoparticles exhibit a variety of shapes including, e.g., spheroidal (e.g., spherical and elliptical), spheroidal with surface dimpling, spheroidal with surface invagination, partially collapsed spheres, completely collapsed spheres (e.g., discoid), irregular shapes, spheroidal shapes with surface features that extend from a general spherical surface or that interrupt a general spherical surface (e.g., spheroidal with fibrous extensions), teardrop-spheroidal, spheroidal with surface protuberances, spheroidal and wrinkled, spheroidal and porous, spheroidal and pitted, and combinations thereof.

The electrosprayed nanoparticles optionally include satellite particles that are substantially spherical but have diameters that are much smaller (e.g., no greater than about 50 nm) than the general population of nanoparticles.

### The Method

The method includes preparing a spray composition. The spray composition can be in a variety of forms including, e.g., solution, dispersion, suspension, and mixtures thereof. Useful methods of preparing the spray composition include, e.g., dissolving the poorly water soluble crystalline compound, a polymer, and any optional additives, in solvent to form a solution. The polymer and the poorly water soluble crystalline compound can be dissolved in the same solvent, in different solvents, and in more than one solvent. When the polymer and the poorly water soluble crystalline compound are dissolved in different solvents, the resulting compositions (e.g., solutions) can be combined before being electrosprayed, can be combined while electrospraying, and combinations thereof. When two or more solvents are used, the solvents preferably are miscible with one another. Other useful methods of preparing the spray composition include, e.g., forming a dispersion, suspension, or emulsion of the poorly water soluble crystalline compound and a carrier, and combining the dispersion, suspension, or emulsion with a solution, suspension, dispersion or emulsion polymer.

The method also includes electrospraying the spray composition using an electrospray system to form nanoparticles from the spray composition. During the electrospraying process the particles in the plume emitted from the cone-jet have a relatively high surface area, the solvent rapidly flashes off of the particles (preferably completely flashes off of the particles), over a relatively short flight path, which concentrates the compound and polymer in the shrinking particles at a time-scale that does not permit re-crystallization. The spray conditions are selected and the ENS system is configured such that the particles have very little residual solvent. The evaporative cooling associated with the extremely rapid solvent evaporation contributes a quenching effect to preserve the particles in the amorphous state.

Useful methods of spraying include, e.g., spraying a single spray composition, spraying a multiple spray compositions, spraying solvents, surfactants, gasses, and combinations thereof, and spraying at least two compositions simultaneously, sequentially, intermittently, continuously, and in combinations thereof.

In some embodiments, the electrospray process is conducted at ambient temperature and pressure and rapidly converts a compound in a crystalline form to an amorphous form. In other embodiments, the spraying occurs at elevated temperatures (i.e., temperatures greater than room temperature) or at temperatures below room temperature.

The nanoparticles can be sprayed onto or into a variety of substrates including, e.g., metal substrates (e.g., stainless steel, gold, silver, aluminum, nickel, and copper substrates and alloys of the same), liquid substrates (e.g., aqueous compositions (e.g., water), organic solvent, simulated body fluid, glycerol, phosphate buffered saline, and combinations thereof), substrates in the form of vessels (e.g., vials), and combinations thereof. The substrate functions to collect the nanoparticles.

The nanoparticles optionally are removed from the substrate. Useful methods of removing the nanoparticles form the substrate include, e.g., using mechanical energy (e.g., scraping the deposited nanoparticles off of the substrate). The sprayed nanoparticles can be in the form of a powder (e.g., a dry powder, dry flakes, non-agglomerated powder, and flowable powder (e.g., free flowing powder)) or made into a powder after removing them from the substrate. The sprayed particles may agglomerate as they are sprayed onto the substrate surface and the method can further include de-agglomerating the particles. De-agglomeration can be achieved by the action that removes the particles from the substrate, e.g., by scraping. De-agglomeration can also be conducted after the particles are removed from the substrate using any suitable method including, e.g., acoustical force (e.g., using a RESODYN acoustic mixer from Resodyn Acoustic Mixers, Inc. (Butte, Mont.), vibrational force, rotational force, mixing, and combinations thereof.

Poorly Water Soluble Crystalline Compound

The method is useful for converting poorly water soluble crystalline compounds to amorphous compounds and is particularly useful for converting a poorly water soluble crystalline drug into its amorphous form. Useful poorly water soluble crystalline drugs include, e.g., azole-containing drugs (e.g., itraconazole), griseofulvin, acetazolamide, acetylsalicylic acid, albendazole, allopurinol, alprazolam, amiloride hydrochloride, amoxicillin, azathioprine, biperiden hydrochloride, carbamazepine, carisoprodol, carvedilol, cimetidine, clarithromycin, clofazimine, clomiphene citrate, clonazepam, dapsone, desogestrel, dexamethasone, diazepam, digoxin, diloxanide furoate, doxycycline, efavirenz, ergotamine tartrate, erythromycin ethyl succinate, estradiol, ethinyl estradiol, famotidine, fluconazole, folic acid, furosemide, gemfibrozil, glibenclamide, glimepiride, glipizide, glyburide, hydrochlorothiazide, ibuprofen, irbesartan, lansoprazole, levodopa, levonorgestrel, levothyroxine sodium, loratadine, lorazepam, meclizine hydrochloride, mebendazole, medroxyprogesterone acetate, mefloquine hydrochloride, metaxalone, methotrexate, methylprednisolone, nalidixic acid, niclosamide, nifedipine, nitrofurantoin, norethindrone, norgestimate, nystatin, olanzapine, phenobarbital, phenytoin, pioglitazone hydrochloride, praziquantel, prednisolone, proguanil hydrochloride, propylthiouracil, pyrantel embonate, pyrazinamide, pyrimethamine, quinine sulfate, reserpine, retinol palmitate, rifampicin, risperidone, ritonavir, rofecoxib, simvastatin, spironolactone, sulfadiazine, sulfamethoxazole, sulfasalazine, theophylline, tamoxifen citrate, temazepam, triamterene, trimethoprim, valproic acid, nonsteroidal anti-inflammatory drugs (e.g., naproxen, indomethacin, celecoxib, meloxicam, and diclofenac), budesonide, asenapine maleate, and combinations thereof), corticosteroids (e.g., triamcinolone acetonide), substance P antagonists (e.g., aprepitant), and combinations thereof.

The method is also useful for converting a variety of other classes of poorly water soluble crystalline compounds into their amorphous forms.

The nanoparticles include at least 10% by weight, at least 20% by weight, at least 25% by weight, at least 35% by weight, at least 40% by weight, at least 45% by weight, at least 50% by weight, no greater than 70% by weight, no greater than 67% by weight, no greater than 60% by weight, no greater than 50% by weight, or even no greater than 45% by weight compound.

Polymer

A variety of amorphous polymers are suitable for use in the spray composition including, e.g., water insoluble polymers, water soluble polymers, FaSSIF soluble polymers, FaSSGF soluble polymers, swellable polymers (e.g., polymers that swell in water, FaSSIF, FaSSGF, and in combinations thereof) and combinations thereof. Useful polymers include polymers that include proton acceptors (e.g., cationic polymers), polymers that include proton donors (e.g., anionic polymers), polymers with surfactant properties, and combinations thereof including, e.g., proton donating copolymers of methacrylic acid and ethyl acrylate, proton accepting copolymers of dimethylaminoethyl methacrylate, butyl methacrylate, and methyl methacrylate, polyvinyl caprolactam-polyvinyl acetate-polyethylene glycol graft copolymer, and combinations thereof. Useful polymer drug combinations include a cationic drug and an anionic polymer, cationic polymer and anionic drug, and combinations thereof.

Suitable amorphous polymers include, e.g., polyvinyl alcohol, polyvinyl acetate, polyvinyl pyrrolidone, hyaluronic acid, alginates, carrageenan, cellulose derivatives (e.g., carboxymethyl cellulose sodium, methyl cellulose, ethyl cellulose, hydroxyethyl cellulose, hydroxypropylmethylcellulose-phthalate, cellulose acetate phthalate, and combinations thereof), non-crystalline cellulose, starch and its derivatives (e.g., hydroxyethyl starch, sodium starch glycolate, and combinations thereof), chitosan and its derivatives, albumen, gelatin, collagen, polyacrylates and polyacrylate derivatives, poly(alpha-hydroxy acids), poly(alpha-aminoacids) and copolymers thereof, poly(orthoesters), polyphosphazenes, poly(phosphoesters), hydroxypropylmethylcellulose-acetate-succinate, and combinations thereof. Useful polymers include functional groups capable of promoting specific interaction with an active agent to help stabilize the amorphous form of the agent.

Suitable water insoluble amorphous polymers include, e.g., polyvinyl acetate, methyl cellulose, ethyl cellulose, non-crystalline cellulose, polyacrylates, polymethacrylates, poly(alpha-hydroxy acids), poly(orthoesters), polyphosphazenes, poly(phosphoesters), and combinations thereof.

Useful commercially available amorphous polymers are available under a variety of trade designations including, e.g., the EUDRAGIT series of trade designations from Evonik (Germany), including EUDRAGIT L100-55 anionic copolymer and EUDRAGIT E100 cationic copolymer, and K30 polyvinylpyrollidone from Sigma-Alrdrich Co. LLC (St. Louis, Mo.) and BASF (Germany).

Particularly useful drug-polymer combinations include, e.g., griseofulvin, itraconazole, aprepitant, and combinations thereof, and SOLUPLUS copolymer; itraconazole and EUDRAGIT L100-55 anionic copolymer based on methacrylic acid and ethyl acrylate, itraconazole, celocoxib, meloxicam, diclofenac, aprepitant and combinations thereof and EUDRAGIT E100 cationic copolymer based on dimethylaminoethyl methacrylate, butyl methacrylate, and methyl methacrylate; naproxen, meloxicam, diclofenac, aprepitant, Indomethacin and combinations thereof and hydroxypropylmethylcellulose-acetate-succinate; and asenapine maleate, indomethacin, celocoxib, deloxicam, diclofenac, aprepitant, and combinations thereof and polyvinylpyrollidone.

The nanoparticles include at least 30% by weight, at least 40% by weight, at least 45% by weight, at least 50% by weight, at least 55% by weight, at least 60% by weight, at least 65% by weight, no greater than about 90% by weight, no greater than about 80% by weight, no greater than about 75% by weight, or even no greater than about 70% by weight polymer.

Solvent

Suitable solvents for use in the spray composition include, e.g., organic solvents, aqueous solvents (e.g., water), and combinations thereof. Useful organic solvents include, e.g., acetic acid, acetone, acetonitrile, methanol, ethanol, propanol, ethyl acetate, propyl acetate, butyl acetate, butanol, N,N dimethyl acetamide, N,N dimethyl formamide, 1-methyl-2-pyrrolidone, dimethyl sulfoxide, diethyl ether, dilsopropyl ether, tetrahydrofuran, pentane, hexane, 2-methoxyethanol, formamide, formic acid, hexane, heptane, ethylene glycol, dioxane, dioxolane. 2-ethoxyethanol, trifluoroacetic acid, methyl isopropyl ketone, methyl ethyl ketone, dimethoxy propane, methylene chloride, n-vinylpyrrolidone, dichloromethane, and combinations thereof. Useful solvent blends include, e.g., ethanol and acetone, methanol and acetone, and methanol and water.

Additives

The spray composition used to form the nanoparticles optionally includes a variety of additives including, e.g., surfactants (e.g., docusate sodium and sodium dodecyl sulfate), excipients, conductivity additives (e.g., ammonium acetate and nitric acid), and combinations thereof.

### Uses

The nanoparticles are suitable for use in a variety of pharmaceutical formulations and pharmaceutical dosage forms including, e.g., tablets (e.g., coated tablets), suppositories, pills, capsules (e.g., soft elastic and hard gelatin capsules), granules, granular powders, powders, aerosols, syrups, solutions, emulsions, suspensions, solutions, and transdermal patches.

Useful pharmaceutical formulations optionally include a variety of additives including, e.g., physiologically acceptable excipients, fillers (e.g., starches, lactose, sucrose, glucose, mannitol, and silicic acid), binders, (e.g., cellulose derivatives (e.g., starch), aliginates, gelatin, polyvinylpyrrolidone, sucrose, gum acacia, tragacanth, alginate, dextran, gelatin, methylcellulose, hydroxypropylmethylcellulose, hydroxypropylcellulose, carboxymethylcellulose, and polyvinylpyrrolidone), humectants (e.g., glycerol), disintegrating agents (e.g., agar-agar, calcium carbonate, sodium carbonate, potato starch and tapioca starch), silicates, quaternary ammonium compounds, adsorbents (e.g., kaolin and bentonite), lubricants (e.g., talc, calcium stearate, magnesium stearate, polyethylene glycols, and sodium lauryl sulfate), preservatives, antibacterial and antifungal agents, stabilizers, complexing agents, antioxidants, flavorings, vitamins, propellants, and combinations thereof.

Useful physiologically acceptable excipients including, e.g., surfactants (e.g., soya lecithin, oleic acid, sorbitan esters, and (e.g., polysorbates) cetyl alcohol, and glycerol monostearate, and magnesium stearate), polyvinylpyrrolidone, monosaccharides (e.g. glucose and arabinose), disaccharides (e.g. lactose, saccharose, maltose, and trehalose), oligo- and polysaccharides (e.g. dextran), polyalcohols (e.g. sorbitol, mannitol, xylitol), cyclodextrines (e.g. alpha-cyclodextrine, beta-cyclodextrine, X-cyclodextrine, methyl-beta-cyclodextrine, hydroxypropyl-beta-cyclodextrine), salts (e.g. sodium chloride and calcium carbonate), and combinations thereof.

Useful propellant gases for aerosols include, e.g., hydrocarbons (e.g., n-propane, n-butane and isobutene) halohydrocarbons (e.g., fluorinated derivatives of methane, ethane, propane, butane, cyclopropane and cyclobutane), and combinations thereof.

### System

The electrospray system includes a single nozzle or a plurality of nozzles positioned in a variety of array configurations including, e.g., a linear array, a circular array, a polygonal array (e.g., a square, a hexagon, and an octagon), offset linear arrays, offset circular arrays, and combinations thereof, that are used to produce nanosize amorphous particles. Throughput can be increased by mounting a plurality of nozzles and operating the nozzles simultaneously. Any suitable number of nozzles can be included in the ENS system including, e.g., at least 4, at least 8, at least 12, at least 15, at least 20, or even at least 25.

In one embodiment, the electrospray system includes a multi-jet, multi-nozzle electrospray system (“ENS”). Each nozzle of the multi jet electrospray system (ENS) simultaneously generates at least 6, at least 12, at least 20, at least 24, at least 192, or even from 6 to 192 jet plumes to produce uniform, submicron amorphous particles of drug and polymer (e.g., particles of GF and SOLUPLUS (SP), a polymer excipient with surfactant-like properties). Each nozzle can be configured to include a number of notches in a variety of configurations as described is, e.g., US 2014/0158787, U.S. Pat. No. 6,093,557, U.S. Pat. No. 6,764,720, and incorporated herein, to produce multiple jets.

The multi-nozzle ENS system can produce powdered formulations of drug and polymer (e.g., GF and SP) that are submicron in scale and form amorphous solid dispersions in a physical state. The multi-nozzle ENS system can operate at ambient temperature and can generate powders of submicron particles in a single step. The ENS process is also capable of producing high drug loading in solid dispersions.

Other useful electrospray devices, systems, and nozzles are described in, e.g., WO 2001/087491, WO 1998/56894, US 2014/0158787, U.S. Pat. No. 7,498,063, U.S. Pat. No. 6,764,720, U.S. Pat. No. 7,247,338, and U.S. Pat. No. 6,093,557, and incorporated herein.

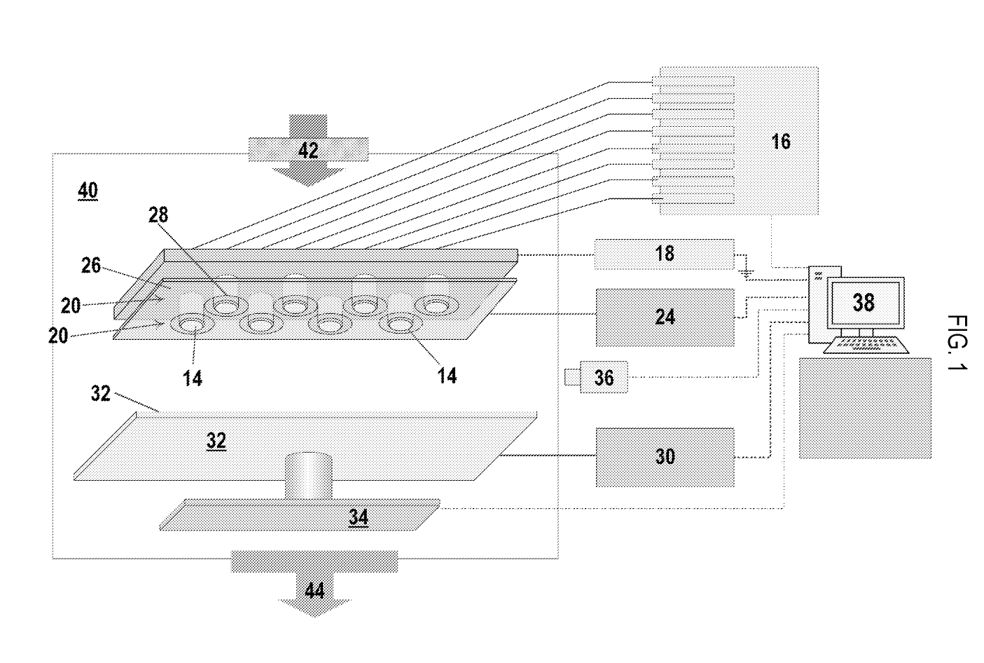

The ENS system preferably includes a plurality of nozzles arranged to achieve an equivalent electric field at all the functional nozzles. One embodiment of an ENS system 10 that includes multiple nozzles 14 disposed in a controlled environment spray chamber 40, a syringe pump 16 in fluid communication with a fluid reservoir that includes a spray composition, two rows 20 of nozzles 14, which are grounded 18 and are arranged in an offset linear pattern with equal spacing between adjacent nozzles 14, a first high voltage power supply 24 electrically coupled to an extractor plate 26 that includes openings 28 there through, a second high voltage power supply 30 electrically coupled to a collector surface 32, which sits on a motor driven stage 34, a camera 36, and a computer 38 is shown in FIG. 1. The controlled environment spray chamber 40 includes air flow 44 which passes through a filter 42. The openings 28 in the extractor plate 26 are positioned to allow spray from the nozzles 14 to pass there through. The computer 38 is coupled to the two high voltage power supplies 24, 30, the syringe pump 16, and a camera 36, which can be used to monitor the droplets, the spray, and nanoparticle formation. The computer can be used to monitor and control the ENS system including, e.g., the high voltage power supplies, the syringe pump, the camera the fluid reservoir, the nozzles, the spray, the collector, and the motor driven stage.

In the arrangement of the nozzles, the electric field of the outermost nozzles can be different from those in the center. Therefore in some embodiments the nozzle array includes “dummy” nozzles having the same geometry as the actual (e.g., working) nozzles. The term “dummy” refers to the fact that no liquid is fed to the dummy nozzles. Dummy nozzles are also referred to herein as non-spraying nozzles. In a two dimensional array, e.g., an array that includes two linear arrays positioned adjacent to one another (an example of which is illustrated in FIG. 1), dummy nozzles can be positioned as the outermost nozzles in the array. Dummy nozzles can be incorporated into the ENS system and positioned as necessary to achieve an equivalent electric field at all the functional nozzles. The ENS system can include any number of dummy nozzles.

In another embodiment, the ENS system includes an extractor rings for each nozzle. By adjusting the diameter of each extractor ring and the distance between the extractor ring and the front end of the nozzle, the onset voltage for electrospray can be reduced, relative to a similarly constructed system without extractors. The base nozzle assembly of the ENS system of FIG. 1 includes two linear 1 by 4 nozzle assemblies where each nozzle and each linear array is mounted in a side by side relationship and the distance between two adjacent nozzles is the same. The system includes an extractor plate with openings that form a ring around each nozzle. The nozzles operate at the same conditions and perform similarly.

The invention will now be described by way of the following examples. All parts, ratios, percentages, and amounts stated in the Examples are by weight unless otherwise specified.

### EXAMPLES

### Test Procedures

Test procedures used in the examples include the following. All ratios and percentages are by weight unless otherwise indicated. The procedures are conducted at room temperature (i.e., an ambient temperature of from about 20° C. to about 25° C.) unless otherwise specified.

### Viscosity Test Method

The viscosity of each spray solution mixture is measured at room temperature using a Viscometer SV-10 (A&D Company, Tokyo, Japan)

### Conductivity Test Method

Conductivity is measured before the electrospray process using an Orion PHuture MMS pH/ORP/Cond 555A multi-parameter meter (Beverly, Mass.). The glass conductivity cell (Orion Model 011010A) has a platinized electrode with a conductivity range of 1 microSiemen per centimeter (μS/cm) to 200 mS/cm. The meter is calibrated before each use with a 100 μS/cm conductivity standard (VWR International LLC, Cat. No. 011008).

### Particle Characterization and Size Measurement by Scanning Electron Microscopy

Stainless steel coupons are used to collect samples of ENS-produced test formulations, using a collection times ranging from 5 seconds and 60 minutes. Scanning electron microscopy is used to evaluate morphology and particle size of ENS powder-coated coupons. Coupons are removed and stored in a desiccator at room temperature until imaging. Sample powder-coated coupons are sputter-coated under vacuum for two minutes with from 10 nm to 15 nm of either gold-palladium or platinum. Sputter-coated samples are then imaged using a Hitachi S-3400N Variable Pressure Scanning Electron Microscope (Hitachi Hitech, Tokyo, Japan) operating at 3 kV, or alternatively, a JEOL 6010Plus/LV Scanning Electron Microscope (JEOL Ltd, Tokyo, Japan).

Particle characterization is performed by imaging samples multiple points on each coupon using various magnifications, ranging from 1000 times to 20,000 times magnification, and noting the shape, size, uniformity and other observations about particle appearance. Particle size is determined from images obtained at 5000 times to 10,000 times magnification and measuring cross-sectional diameter of 100 fully visible particles sampled from each quadrant using a large video monitor and pen tablet to facilitate measurements, with diameters scaled using the micron size marker provided by the instrument for each image magnification level.

### Particle Size Measurement in Liquid by Nanoparticle Tracking Analysis Test Method

The particle size measurements in liquid by nanoparticle tracking analysis (NTA) is performed using a NanoSight NS500 (NanoSight-Malvern, Minton Park, Amesbury, Wilshire, United Kingdom). Each sample of powder, with its weight normalized to provide 200 μg of the compound of interest, is transferred to duplicate microcentrifuge tubes (VWR, catalog No. 87003-29). A 1.0 mL aliquot of FaSSIF is then added to each tube. The samples are vortexed for 10 minutes and then centrifuged for 15 minutes at 16,000×g in a microcentrifuge (MC 1400 Microcentrifuge, Hoefler Scientific Instruments, San Francisco, Calif.). A 0.5 mL aliquot of the supernatant of each sample is pooled in a separate microcentrifuge tube, and loaded into the instrument chamber. An autosampler then withdraws an aliquot and introduces it into the viewing chamber. For each aliquot, a 15 second (sec) video of the laser-illuminated particle tracks in the field of view of the microscope objective lens is captured using a scientific digital camera. Nine separate aliquots of each sample are measured. Analysis for particle size and distribution is performed using Nanoparticle Tracking Analysis software suite (Malvern Instruments, Ltd., Malvern, United Kingdom), which processes the captured video images frame-by-frame, calculates particle size on a particle-by-particle basis, plots the size distribution, and calculates the size mean, mode and standard deviation.

### Particle Size Analysis in Biorelevant Media by Laser Diffraction Test Method

Particle size measurements by laser diffraction are analyzed using a Horiba LA-960 laser diffraction analyzer fitted with a 10 mL sample vessel with magnetic stir bar. A 50 mL aliquot of FaSSGF medium (which is prepared according to the Dissolution in Biorelevant Media with pH Transition Test Method) and a magnetic stirrer are placed in a 100 mL Erlenmeyer flask, which is then placed on a magnetic stir plate. All solutions are at room temperature. The refractive index (RI) used for the calculations is estimated from the published values and relative amounts of the major solid components. For ITZ the RI is 1.46, which is based on the published values, which ranged from a high of 1.62 for ITZ and a low of 1.33 for water. After wetting a 20 mg sample of test formulation with an aliquot of FaSSGF, the resulting slurry is added to the remaining FaSSGF solution. The initial 10 mL sample is transferred to the 10 mL sample vessel and preliminary transmittance readings are taken, further diluting the sample with additional FaSSGF necessary to increase the transmittance into the optimal range. Measurements are taken and particle size distribution of the sample and subpopulations are recorded. Transition Medium is added to the FaSSGF test solution in the external flask at 30 min. The sample vessel is washed and new sample from the resulting FaSSIF test solution is transferred into the sample vessel. Transmittance values are adjusted if required by dilution and repeated measurements are taken over the next 3 hours. By conducting the dissolution procedure external to the instrument, appropriate transmittance values can be achieved by dilution, and when necessary, adding a larger quantity of test formulation at the beginning of the FaSSGF phase if the transmittance levels are noted to increase as the dissolution study progresses. Measurements are analyzed using the LA-960 software to determine D50, D90 and D10 size values for the particle population as a whole and for subpopulations when two or more are present.

### Differential Scanning Calorimetry (DSC) Test Method

Thermal analysis is conducted using a TA Instruments Model Q200 DSC (New Castle, Del.) equipped with an RCS-90 refrigerated cooling system. Samples, in an amount of 3.5 mg±1.0 mg, are weighed into TA Instruments T-Zero aluminum pans and closed with lids. Samples are heated at a ramp rate of 3° C./min from −20° C. to 225° C. Nitrogen is used as the purge gas at a flow rate of 50 mL/min. All data analyses are performed using TA Universal Analysis 2000 software (TA Instruments, New Castle, Del.). T-Zero cell constant and temperature/enthalpy calibrations are conducted with the use of sapphire and indium standards, respectively.

### Thermal Gravimetric Analysis (TGA) Test Method

Thermal gravimetric analysis (TGA) is conducted using a Model Q500 TGA thermal gravimetric analysis instrument from TA Instruments (New Castle, Del.). Samples, in an amount of 20 mg±4 mg, are weighed into TA Instruments T-Zero platinum pans for analysis. Samples are heated at a ramp rate of 20° C./min from 40° C. to 250° C. Nitrogen is used as the purge gas at flow rates of 40 mL/min and 60 mL/min for the balance and sample, respectively. Temperature is calibrated to the Curie points of Alumel and nickel. All data analyses are performed using TA Universal Analysis 2000 software from TA Instruments (New Castle, Del.).

### X-Ray Powder Diffraction (XRD) Test Method

X-ray powder diffraction is performed at room temperature using a Bruker D8 Discover using a Cu Kα radiation, point source filtered with a graphite monochromator and a Bruker Hi-Star two-dimensional x-ray detector (Bruker, Karlsruhe, Germany). Powder samples are pressed onto a sample holder made of single-crystal quartz cut off-axis. The reflected intensity is collected at 0.04° resolution from 2Θ=2° to 62° with an acquisition time of 20 minutes. Data analysis is performed using the JADE 8.0 software package (Materials Data Inc., Livermore, Calif.) with whole pattern fitting. The test method has a detection limit of 2% crystallinity.

### Fourier Transform Infrared Spectroscopy (FTIR) Test Method

Fourier transform infrared spectroscopy spectra are collected as the mean of 32 scans at a resolution of 4 cm⁻¹ using a Nicolet iS10 FT-IR Spectrometer (Thermo Scientific Nicolet, Waltham, Mass.). Powder samples are placed directly on the diamond crystal of a Thermo Scientific Smart iTR single bounce, attenuated total reflectance (ATR) sampling accessory (Thermo Fisher Scientific Inc., Waltham, Mass.).

### Dissolution in Biorelevant Medium Using Non-Sink Condition Test Method

A modified low-volume shaker flask method is used to assess the dissolution behavior of samples under supersaturated conditions. A 75 mL aliquot of FaSSIF, adjusted to pH 6.5, is added to each of six 150 mL Erlenmeyer flasks, which are then stoppered. The liquid is then heated to and maintained at 37° C.±1° C. using a benchtop incubated orbital shaker with a multi position stir plate (Forma Scientific 4520, Thermo Fisher Scientific, Waltham, Mass.). A 38 mm×9 mm stir bar is placed in each flask and stirring is started at 150 rotations per minute (RPM). A control sample, containing the compound of interest, and experimental samples are added to separate flasks in amounts sufficient to provide an amount of the compound of interest equivalent to the control sample. At each of 5 min, 15 min, 20 min, 30 min, 45 min, 60 min, 120 min, 240 min, and 300 min, 3 mL of dissolution media is removed from each flask, and immediately filtered through a 13 mm, 0.2 μm nylon filter with glass prefilter (EMD Millipore, Billerica, Mass.) after first discarding the first 2 mL of the sample. The filtered sample is then diluted 1:1 with acetonitrile to prepare the sample for analysis by high performance liquid chromatography (HPLC) with UV detection (Agilent 1200 LC, Agilent Technologies, Santa Clara, Calif.).

The HPLC analysis is performed using an Ace C18, 2.1×150 mm, 3 μm chromatographic column maintained at 30° C. The mobile phase is operated under isocratic flow of 0.3 mL/min and consists of 0.1% formic acid in water and 0.1% formic acid in acetonitrile in a ratio of 44:56 v/v. The injection volume is 5 μL. Quantification is performed based on linear calibration curve ranging from 10 ng/mL to 1000 ng/mL using peak response ratio.

### Caco-2 Monolayer Test Method

Caco-2 monolayers are prepared in Millipore 96-cell Caco-2 plates and are used after a 3-week differentiation period. The monolayers separate the apical, or donor well, from the basolateral, or recipient well. The buffer used for the apical side is 1.98 g/L glucose in 10 mM HEPES, Hank's Balanced Salt Solution (HBSS) lx, with calcium, magnesium, without phenol red (HyClone, Logan, Utah) pH 7.4. The impermeable dye Lucifer yellow (in an amount of 100 μM) is added to test monolayer integrity. The basolateral side buffer is buffer, pH 7.4, without dye. Samples of test powder equivalent to 200 μg of the compound of interest are dissolved in 1 mL buffer, vortexed for 5 sec, sonicated for 10 min, vortexed an additional 5 sec, and then centrifuged for 15 min to reduce undissolved aggregates. The amount of test powder is selected to assure that if the powder was completely dissolved, the amount is sufficient to achieve ˜12 to 13 times the saturation concentration of the compound of interest previously measured in HBSS (15 μg/mL), in other words, a supersaturated solution. In this example, the amount of test powder would be capable of generating a concentration as high as 180 to 195 μg/mL if it were completely dissolved and remained in solution while the saturated concentration of the compound of interest was 15 μg/mL. Where the compound of interest is not a substrate for p-glycoprotein (pGP), B→A efflux is not measured.

Buffer test samples are added to apical wells at 1 h after preparation and buffer alone is added to the basolateral side. Reference compounds in the test plate include warfarin, ranitidine, and talinolol. The plates are incubated for 2 hours (h) before removing the receiver side buffer for analysis. The plates are assumed to be intact if there is no increase in fluorescence from leaking Lucifer yellow dye from the apical side. Individual cell monolayers are assumed intact if there is no increase in fluorescence in aliquots taken from the basolateral wells. Concentrations of the compound of interest in apical and basolateral well samples are measured by an LC/MS/MS with an Agilent 6410 mass spectrometer (MS) and 1200 HPLC with a CTC PAL chilled autosampler, controlled by MassHunter software (Agilent Technologies, Santa Clara).

The peaks of the compound of interest are separated on a C18 reverse phase HPLC column (Agilent) using an acetonitrile-water gradient system and analyzed by MS positive ESI ionization in MRM mode using parent ion 352.8 m/z and daughter ion 165 m/z. The apparent permeability of the compound of interest (Pₐₚₚ) is calculated according to the following equation:

$${P}_{app} = \frac{\frac{d Q}{d t}}{{C}_{o} \cdot A}$$

where A is the area of each monolayer in units of cm², Cₒ is the initial concentration of the compound of interest in units of μg/mL, and dQ/dt is the permeation rate, or flux, in units of μg/s.
Dissolution in Buffer Media with pH Transition Test Method

Dissolution testing, based on pH change from acidic-to-neutral media, is performed according to USP 29 Apparatus 2 Guidelines (United States Pharmacopeial Convention. Chapter <711> Dissolution, Stage 6 Harmonization, Official Dec. 1, 2011. USP website: http://www.usp.org/sites/default/files/usp_pdf/EN/USPNF/2011-02-25711DISSOLUTION.pdf).

Where specified below, the USP method was modified to use smaller 100 mL glass vessels with matching sized paddles operated at a speed of 100 rpm in buffer media using a VanKel 7000 Dissolution Tester (VanKel Technology Group, Gary, N.C.).

Dissolution in Biorelevant Media with pH Transition Test Method

Dissolution testing, based on pH change from acidic-to-neutral media, is performed according to USP 29 Apparatus 2 Guidelines (United States Pharmacopeial Convention. Chapter <711> Dissolution, Stage 6 Harmonization, Official Dec. 1, 2011. USP website: http://www.usp.org/sites/default/files/usp_pdf/EN/USPNF/2011-02-25711DISSOLUTION.pdf).

Where specified below, the USP method was modified to use smaller 100 mL glass vessels with matching sized paddles operated at a 100 mL glass vessels with paddle speed of 100 rpm in biorelevant media using a VanKel 7000 Dissolution Tester (VanKel Technology Group, Gary, N.C.).

Biorelevant media is prepared using FaSSIF, FeSSIF & FaSSGF Powder (biorelevant.com) a commercial source of sodium taurocholate and lecithin, hereafter abbreviated “SIF Powder.” Fasted state simulated gastric fluid (FaSSGF) and Fasted state simulated intestinal fluid (FaSSIF) are prepared using the formula provided by the vendor at http://biorelevant.com/fassif-fessif-fassgf-dissolution-media/fasted-fed-state-simulated-intestinal-gastric-fluid/how-to-make/, after specifying the desired volume quantity of medium to be prepared. For example, for 500 mL of FaSSGF, the test medium contains sodium chloride, 1 g, and SIF Powder, 30 mg. Solids are dissolved in approximately 480 mL deionized water. The FaSSGF solution is adjusted to a final pH of 1.6 using concentrated hydrochloric acid, and brought to a final volume of 500 mL by adding deionized water.

For two-stage dissolution testing, which consists of a gastric stage followed by an intestinal phase, the compound of interest is first added to a volume of FaSSGF that is equivalent to ¾ of the final test volume. For example, if the test vessel is 100 mL, the volume of FaSSGF is 75 mL. After allowing dissolution to proceed for 2 hours or another specified time interval, 25 mL of a Transition Medium is added to create a final volume of 100 mL in this example, with a final pH of 6.5. The final medium is equivalent in composition to FaSSIF, as per the formula provided at the above link at biorelevant.com.

The Transition Medium is prepared first by making Transition Buffer, 500 mL, which contains sodium chloride, 9.37 g, sodium hydroxide, 2.5 g, sodium phosphate monobasic dihydrate, 8.94 g. Solids are dissolved in approximately 480 mL of deionized water, adjusted to a final pH of 10.45 with concentrated sodium hydroxide solution, and then brought to a final volume of 500 mL by adding deionized water. To make 50 mL of Transition Medium, SIF Powder, 439 mg, is dissolved in an aliquot of Transition Buffer that is brought to a final volume of 50 mL. The Transition Buffer will have a slightly iridescent quality after addition of the SIF Powder. Larger quantities are prepared as required, maintaining the relative amounts each of Transition Buffer and SIF Powder.

The dissolution test is performed by weighing the test formulation containing the test compound, adjusting weights to achieve an equivalent amount of test compound across the various test formulations. The weight of the compound added is sufficient to achieve non-sink conditions, at a specified multiple of (20×) of its equilibrium solubility in the test medium. The test formulation is wet, drop by drop in an agate or glass mortar and pestle, as per the ISO Standard 14887:2000E, Dispersing Procedures for Powders in Liquids, using a 1-3 mL aliquot of FaSSGF from the first test vessel to create a slurry. Starting time for the dissolution test is recorded with the addition of the first drop. Preparation of the slurry is completed within 2-5 minutes, after which the resulting 1-3 mL of slurry is transferred to the test vessel containing FaSSGF and the test continued. After the desired dwell time in FaSSGF, the appropriate volume of Test Medium for the test vessel size is added quickly, and the test continued for the desired dwell time in the resulting FaSSIF.

### Sample Preparation

### Electrospray Spray System (ENS System) and Process (ENS Process)

The core elements of the ENS system include nozzles, a gantry for mounting nozzles, a syringe pump (useful examples of which include 2-syringe PHD-22/2000 infusion/withdraw pump, Harvard Apparatus, Holliston, Mass.) and a New Era Pump Systems OEM 6-syringe model (Farmingdale, N.Y.), a high-voltage (e.g., 30 kV) power supply (Spellman High Voltage Electronics, Hauppauge, N.Y.), and a collector substrate, a diagram showing an example of a suitable electrospray system is shown in FIG. 1. The system can also be configured with an extractor plate in the form of an aluminum plate with holes that are concentric with each nozzle. The extractor plate is mounted on the gantry between the nozzles and substrate. An independent high-voltage power supply is electrically coupled to the extractor plate. The extractor plate provides supplemental high voltage and enables the particle flight path to be lengthened to facilitate particle drying. In the examples below in which an extractor plate was used, the operating voltage is described.

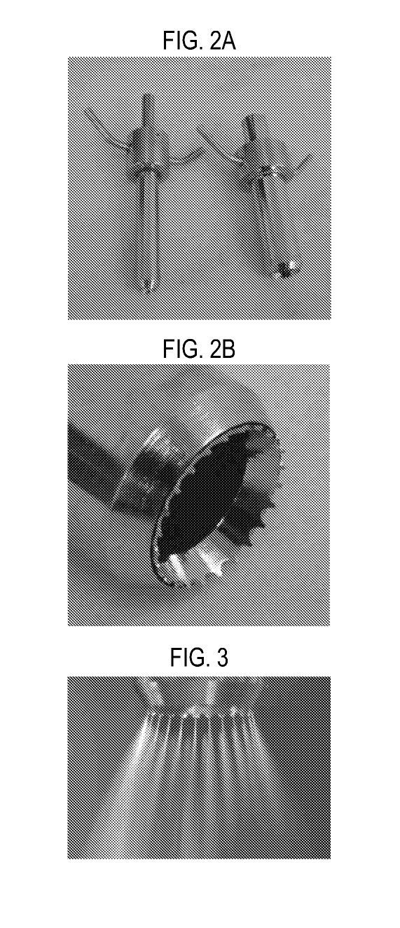

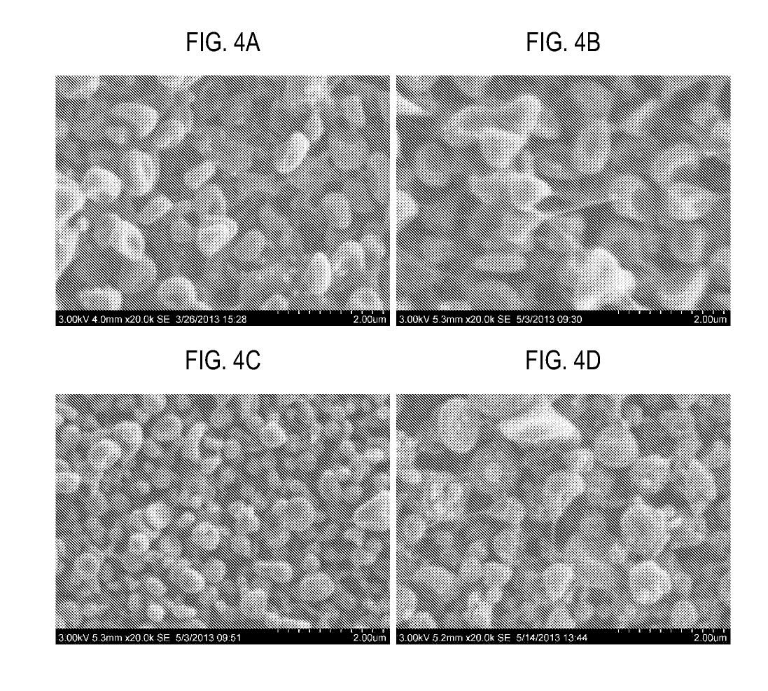

Dry powder nanoformulations were produced using an ENS system from Nanocopoeia, Inc. (St. Paul, Minn., US) configured with from one to eight nozzles. Two nozzle sizes were used, one with 12 cone jets (referred to herein as D12 nozzle) and the other with 24 cone jets (referred to herein as D24 nozzle). The specific nozzle size used in an example is specified in the example. The nozzles are shown in FIGS. 2A, 2B, and 3. The photographs of the D12 and D24 nozzles show the circular form of the nozzles and the boundary of the inner notched ring and the outer tapered cylinder. The spray solution was fed from the top of the nozzle and flows as a sheet between the inner and outer cylinders, terminating at the edge of the notched ring. For each example formulation, the respective spray solution was delivered to the multi jet nozzle array using a constant feed syringe pump. After priming the system with solution, the voltage was gradually increased to about 30 kV to establish the electric field between the nozzles and the substrate, with the final operating voltage adjusted to a level at which a stable cone jet operation was achieved. A photograph of a stable cone jet operation is shown in the photograph of FIG. 3, in which a D24 nozzle spraying in cone-jet mode emits 24 independent plumes from the notched ring of the nozzle. The spray was directed toward a collector substrate positioned to receive the resulting nanoparticles.

### Materials

(+)-griseofulvin (GF) powder (Alfa Aesar, Haysham, United Kingdom).

SOLUPLUS (SP) polyvinyl caprolactam-polyvinyl acetate-polyethylene glycol grafted copolymer powder (BASF, Ludwigshafen, Germany).

Dioctyl sulfosuccinate sodium salt (docusate sodium (DS)) (Acros Organics, Geel, Belgium)

Sodium dodecyl sulfate (SDS) (Sigma-Aldrich, St. Louis, Mo.).

200 proof HPLC/spectrophotometric grade ethanol (EtOH) (Sigma-Aldrich Corporation, St. Louis, Mo.)

BDH-ACS reagent grade acetone (Ace) (VWR, Chicago, Ill.)

HPLC grade acetonitrile (Macron Fine Chemicals, Center Valley, Pa.)

HPLC grade water (Omnisolv, Billerica, Mass.),

96+% grade formic acid (Alfa Aesar, Woodhill, Mass.).

FaSSIF, FeSSIF & FaSSGF Powder (Biorelevant.com, London, United Kingdom) (SIF powder)

Transition media, Transition Buffer, Fasted-State Simulated Gastric Fluid (FaSSGF) and Fasted-State Simulated Intestinal Fluid (FaSSIF) are prepared as described in the Dissolution in Biorelevant Media with pH Transition Test Method

Itraconazole powder (Tokyo Chemical Industry, Co. Ltd., Tokyo, Japan)

SPORANOX 100 mg ITZ capsules (Janssen Pharmaceuticals, Inc., Titusville, N.J.)

EUDRAGIT L100-55 (L100-55) anionic polymer (Evonik Industries AG, Essen, Germany)

EUDRAGIT E100 (E100) cationic polymer (Evonik Industries AG, Essen, Germany)

GR grade dichloromethane and trisodium phosphate (EMD Serono, Inc., Rockland, Mass.).

The biorelevant media identified as FaSSIF, FeSSIF and FaSSGF have the following compositions and properties, where mM is millimolar, mOsm/kg is milliosmoles per kilogram, and mM/ApH is millimolar per change in pH.

| | FaSSGF | FaSSIF | FeSSIF |
|---|---|---|---|
| Sodium taurocholate (mM) | 0.08 | 3.0 | 15 |
| Lecithin (mM) | 0.02 | 1.75 | 3.75 |
| Sodium chloride (mM) | 34.2 | 105.9 | 203.2 |
| Sodium hydroxide (mM) | 0 | 8.7 | 101.0 |
| Hydrochloric acid (mM) | 25.1 | 0 | 0 |
| Monobasic sodium phosphate | 0 | 28.4 | 0 |
| (mM) | | | |
| Acetic acid (mM) | 0 | 0 | 144.1 |

Characteristic parameters

| | | | |
|---|---|---|---|
| pH | 1.6 | 6.5 | 5.0 |
| Osmolatity (mOsm/kg) | 120 ± 2.5 | 270 ± 10 | 670 ± 10 |
| Buffer capacity (mM/ΔpH) | — | 10 | 76 |

### Controls 1-7

Control 1 was the powdered crystalline form of griseofulvin as supplied by the manufacturer.

Control 2 was the powdered crystalline form of griseofulvin dissolved in a solvent blend of ethanol and acetone (having a volume to volume (v/v) ratio of 3:2 ethanol:acetone). The solvent was allowed to evaporate before testing.

Control 3 was the powdered crystalline form of griseofulvin sprayed according to the ENS process.

Control 4 was SOLUPLUS polymer in powder form.

Controls 5 and 7-12 were physical mixtures prepared by mixing the griseofulvin (GF), SOLUPLUS polymer (SP), and surfactant, where present, in the amounts, in % by weight, and ratios specified in Table 1, at room temperature, in a ceramic mortar and pestle with gentle force for about 5 minutes or until a fine and uniform mixture was obtained. The resulting mixtures were then transferred to glass vials, capped and stored in a desiccator at room temperature.

Control 6 was first prepared according to the method described in Control 5 and subsequently dissolved in 3:2 ethanol:acetone. The solvent was then allowed to evaporate before testing.

### Examples 1-7

Examples 1-7 were dry powders prepared according to the ENS process using a D12 nozzle. Spray solutions used to form the dry powders of Examples 1-7 were prepared by dissolving the solids of each sample formulation, in the amounts specified in Table 1 (in % by weight), in a 3:2 ethanol:acetone solvent blend. The spray solutions were then sprayed using the above-described ENS System and ENS Process to obtain the dry powders of Examples 1-7 with the following exceptions.

Small polished stainless steel coupons were positioned in various locations on the collector plate so as to obtain samples of the sprayed composition for imaging. The product sprayed from the nozzles was collected on the stainless steel plates located approximately 15 cm beneath the nozzle array in the form of a dry powder. The dry powder product was gently removed from the collector plate by scraping, placed into a glass vial, and stored in a desiccator until characterization and dissolution testing.

TABLE 1

| | | | | | Component | | Solution |
|---|---|---|---|---|---|---|---|
| | | | | | Ratio in | | Conduc- |
| | | | | | Solvent | | tivity |
| Sample | GF | SP | DS | SDS | Formulations | Process | μS/cm |
| Control 1 | 100 | 0 | 0 | 0 | NA | Powder | NA |
| Control 2 | 100 | 0 | 0 | 0 | NA | SE | NA |
| Control 3 | 100 | 0 | 0 | 0 | 1 | ENS | 1.2-1.8 |
| Control 4 | 0 | 100 | 0 | 0 | NA | Powder | NA |
| Control 5 | 50 | 50 | 0 | 0 | NA | Physical | NA |
| | | | | | | Mixture | |
| Control 6 | 50 | 50 | 0 | 0 | NA | SE | NA |
| Example 1 | 50 | 50 | 0 | 0 | 1:1 | ENS | 1.4 |
| Control 7 | 47.6 | 47.6 | 4.8 | 0 | NA | Physical | NA |
| | | | | | | Mixture | |
| Example 2 | 47.6 | 47.6 | 4.8 | 0 | 1:1:0.1 | ENS | 70.3 |
| Control 8 | 47.6 | 47.6 | 0 | 4.8 | NA | Physical | NA |
| | | | | | | Mixture | |
| Example 3 | 47.6 | 47.6 | 0 | 4.8 | 1:1:0.1 | ENS | 111.3 |
| Control 9 | 66.7 | 33.3 | 0 | 0 | NA | Physical | NA |
| | | | | | | Mixture | |
| Example 4 | 66.7 | 33.3 | 0 | 0 | 1:0.5 | ENS | 1.4 |
| Control 10 | 50 | 50 | | | NA | Physical | |
| | | | | | | Mixture | |
| Example 5 | 50 | 50 | | | 1:1 | ENS | 1.4 |
| Control 11 | 33.3 | 66.7 | | | NA | Physical | |
| | | | | | | Mixture | |
| Example 6 | 33.3 | 66.7 | | | 1:2 | ENS | 1.5 |
| Control 12 | 16.7 | 83.3 | | | NA | Physical | |
| | | | | | | Mixture | |
| Example 7 | 16.7 | 83.3 | | | 1:5 | ENS | 1.9 |

GF = griseofulvin
SP = Soluplus
DS = docusate sodium
SDS = sodium dodecyl sulfate
μS/cm = microsiemens per centimeter

The powders of Examples 1-7 were then tested according to the Particle Characterization and Size Measurement By Scanning Electron Microscopy Test Method, the Particle Size Measurement In Liquid By Nanoparticle Tracking Analysis Test Method, Differential Scanning calorimetry (DSC) Test Method, Thermal Gravimetric Analysis (TGA) Test Method, X-ray diffraction (XRD) Test Method, Fourier Transform Infrared Spectroscopy (FTIR) Test Method, and Dissolution In Biorelevant Medium Using Non-Sink Condition Test Method, with the exception that the XRD test method was performed at room temperature using a Rigaku MiniFlex 600, data was obtained using a Cu Kα radiation source and a D/teX Ultra High-Speed 1D Detector, powder samples were pressed onto silicon zero-background sample holder with a 0.1 mm indent, the reflected intensity was collected at 0.02° resolution from 2Θ=5° to 55° with an acquisition rate of 5.0 degrees/min, and data analysis was performed using the commercial software PDXL2 (Rigaku). The results are described below.

For the Dissolution in Biorelevant Medium Using Non-Sink Condition analysis, a 24 mg sample of Control 1, i.e., griseofulvin powder, and an amount of Examples 1-7 sufficient to provide an amount of griseofulvin equivalent to the amount of Control 1, i.e., 24 mg, was added to each flask. Quantification was performed based on linear calibration curve ranging from 10 to 1000 ng/mL using peak response ratio. The lower limit of detection was 10 ng/mL.

Particle Characterization of ENS-Processed Powders

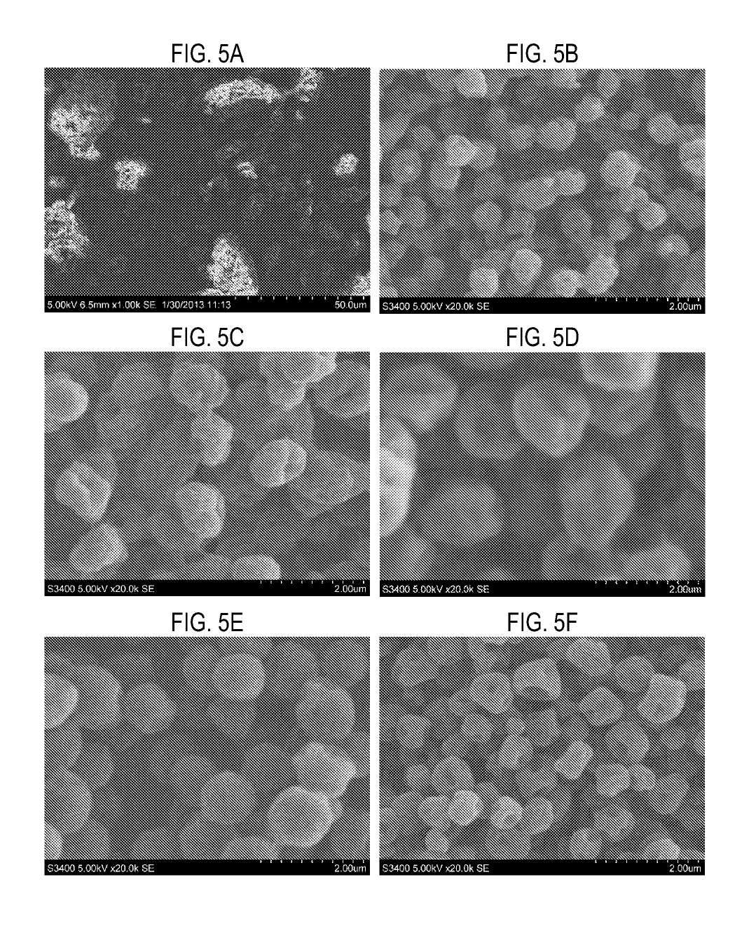

SEM microscope images show particles for (a) Control 3 (GF ENS), (b) Example 1 (GF:SP-1:1-ENS), (c) Example 3 (GF:SP:SDS-1:1:0.1-ENS), and (d) Example 4 (GF:SP:DS-1:1:0.1-ENS) all at ×20,000 magnification, as shown in FIGS. 4A-4D, respectively. The arrows are pointing to examples of smaller satellite particles. Despite their numbers, the relative mass of the satellite particles in the powder is small. Particle size was notably smaller in the powder images of GF:SP:SDS-1:1:0.1 (FIG. 4C), which also include satellite particles. Without being bound by theory, the present inventors believe the satellite particles form due to particle break-up when surface charge exceeds the Rayleigh limit.

### Control 8 and Examples 8-11

The powders of Controls 8 and Examples 8-11 were produced using the ENS System equipped with an eight-nozzle D24 array according to the ENS Process. The spray solutions of Examples 8-11 were prepared as described above with respect to Examples 4-7 with the exception that the amount of GF and SP polymer used in each formulation was sufficient to produce the following GF:SP weight to weight ratios: Example 8 GF:SP 1:0.5, Example 9 GF:SP 1:1, Example 10 GF:SP 1:2, and Example 11 GF:SP 1:5.

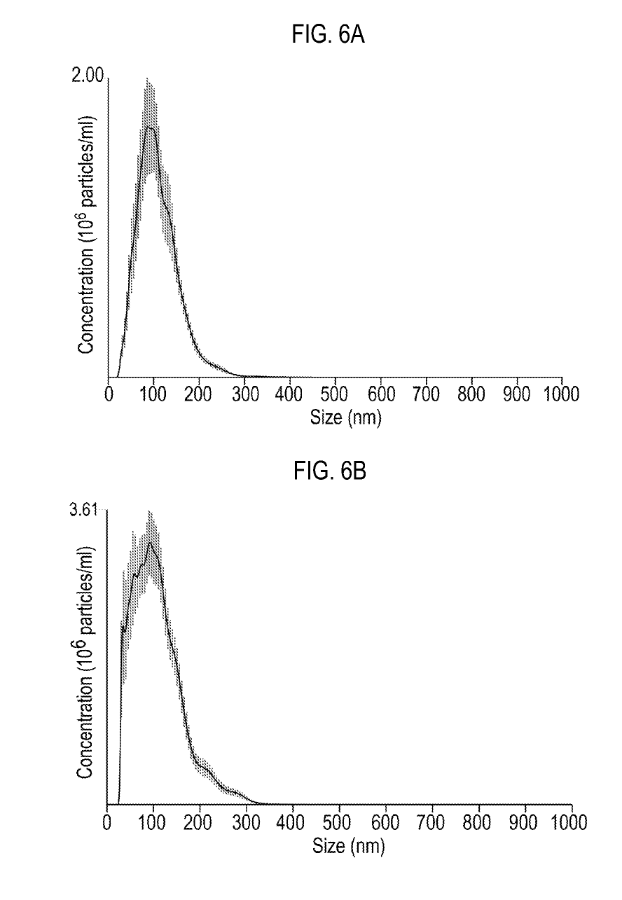

FIGS. 5A-5F are SEM images of GF:SP-ENS powders collected under one of the operating D24 nozzles, GF powder crystals (Control 1), and GF-ENS powder (Control 8). Except for the GF-ENS (Control 8), where particles are approximately 200-250 nm in diameter, particles are larger and more discrete. Some dimpling in surface morphology is visible, consistent with the surface solvent evaporating rapidly so that the particles skin over before contents dry. Particles are irregular but similar in appearance within each set. FIGS. 5A-5F are SEM images of GF formulations with varying ratios of SP: 5A Control 1: GF powder, as received, D24-nozzle ENS-processed formulations of 5B GF (Control 8), 5C GF:SP-1:0.5 (Example 8), 5D GF:SP-1:1 (Example 9), 5E GF:SP-1:2 (Example 10), and 5F GF:SP-1:5 (Example 11), at higher magnification. Electrospray operating parameters listed beneath images 5A-5F include conductivity (a), spray fluid flow rate (F), voltage (V), extractor voltage (EV), and nozzle-to-substrate distance (D).

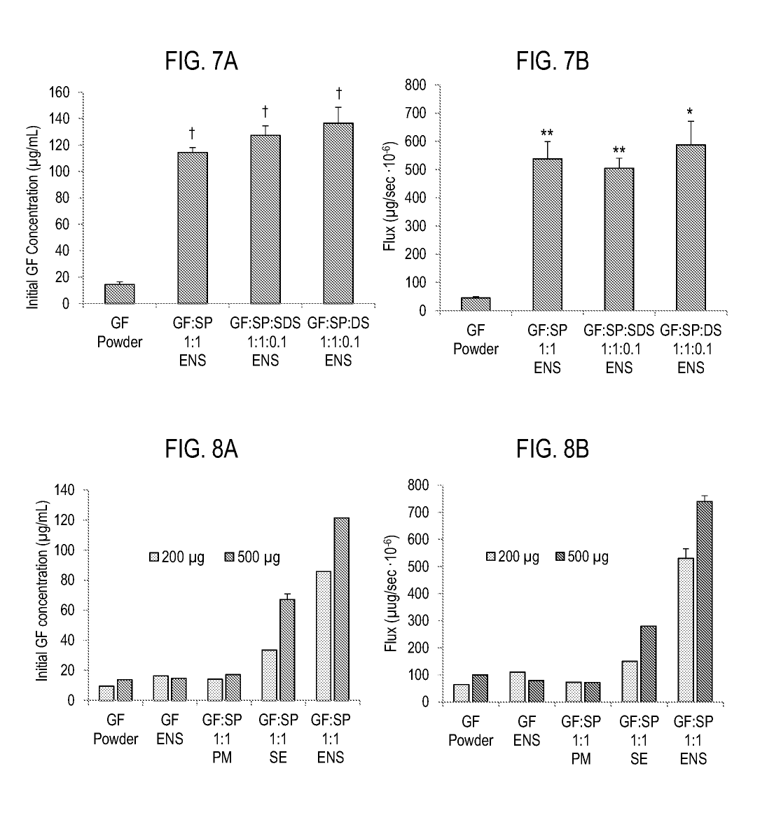

FIGS. 6A and 6B show particle size distributions in FaSSIF for SP (using Nanoparticle Tracking SOLUPLUS powder) alone (the mean particle size is 111±44 nm and the mode particle size is 95 nm), dissolved as received without ENS processing, and GF:SP-1:2-ENS (the mean particle size is 109±51 nm and the mode particle size is 73 nm) measured Analysis. Peaks represent average particle size and concentration in test medium, from nine successive measurements. Gray error bars represent ±1 standard error of the mean. The concentration of SP in solutions resulting from the dissolution of the various powders used in this study was at least 100 mg/L (1% w/v), well above the reported critical micelle concentration of 7.6 mg/L.

### Caco-2 Monolayer Analysis

Examples 1-7 were tested according to the Caco-2 Monolayer Test Method with the exception that the samples were packed in sealed vials with silica desiccant and sent by overnight courier to Apredica, Inc. (Watertown, Mass.), where the Caco-2 monolayer experiments were performed. The amount of test powder was selected to achieve from ˜12 to 13 times the saturation concentration of GF previously measured in HBSS (i.e., 15 μg/mL). GF is not a substrate for p-glycoprotein (pGP); therefore, B→A efflux was not measured.

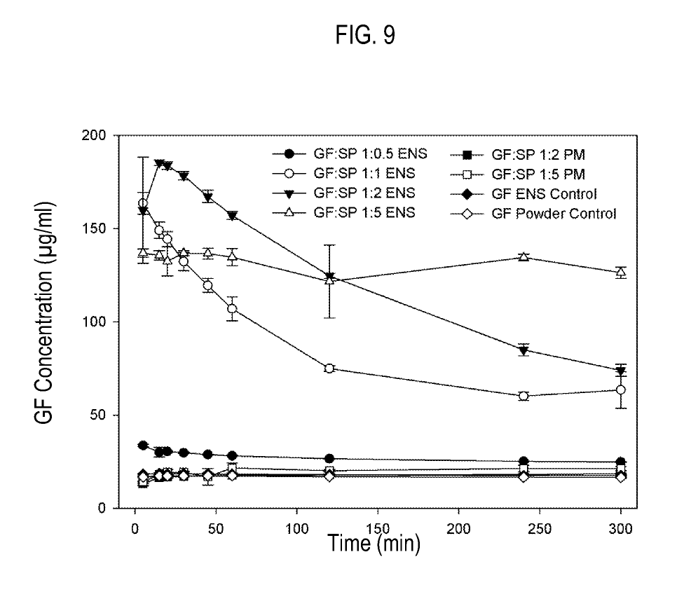

All powders used for the Caco-2 tests were produced by the D12 nozzle. FIGS. 7A and 7B compare initial concentrations for ENS-sprayed powders (Examples 1, 2, and 3), with (Examples 2 and 3) and without (Example 1) surfactant, and GF flux values for each of Control 1 and Examples 1, 2, and 3, which showed increases in similar scale to the increased initial concentrations. The ENS-sprayed powders (i.e., Examples 1, 2, and 3) had the highest overall GF flux across the monolayer for those formulations, which resulted in higher initial drug concentrations in the apical cells. The apparent permeability was higher for all of the ENS-sprayed powders as demonstrated by Table 2.

TABLE 2

| Sample | Formulation | Pₐₚₚ (*10⁻⁶ cm/s) |
|---|---|---|
| Control 1 | GF | 28.0 ± 2.5 |
| Example 1 | GF:SP-1:1 | 42.5 ± 0.4 |
| Example 2 | GF:SP:DS | 39.1 ± 1.7 |
| Example 3 | GF:SP:SDS-1:1:0.1 | 36.2 ± 2.3 |

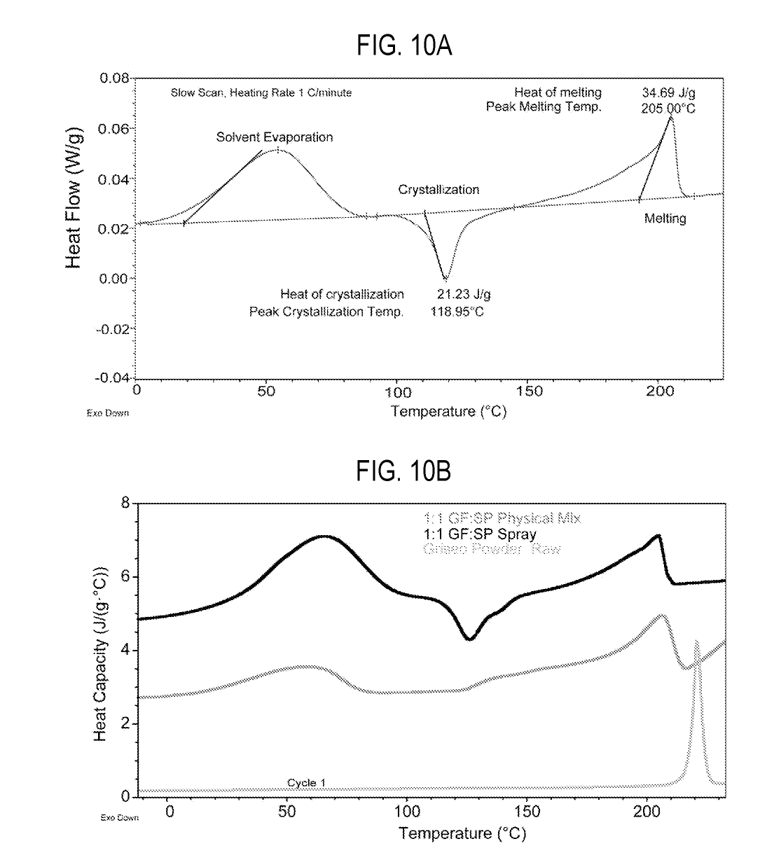

FIGS. 8A and 8B compare initial concentrations and flux values for the test materials with GF unprocessed powder serving as the control (Control 1). Control 3 (GF-ENS) and Control 4 (GF:SP-1:1-PM) performed similarly to unprocessed GF powder. When GF and SP were combined in solvent and evaporated to solid Control 5 (GF:SP-1:1-SE), the initial concentration and flux increased at least 3-fold. Example 1 (GF:SP-1:1-ENS) showed further increases. Increasing the loading dose of GF from 200 μg to 500 μg was associated with increases for the ENS-produced powder but not for the other preparations. The initial concentration and flux increased by approximately 50%, but less than the 150% increment in dose. These results demonstrate the effect of combining the GF and SP in solvent solution, where the interaction of the drug and polymer is at the molecular level, while the interaction is only at the particle level in the physical mixture. Unlike the solid mixture obtained by evaporating the solvent over a few hours, the ENS-produced powder dried very rapidly.

Dissolution Profiles in Biorelevant Media

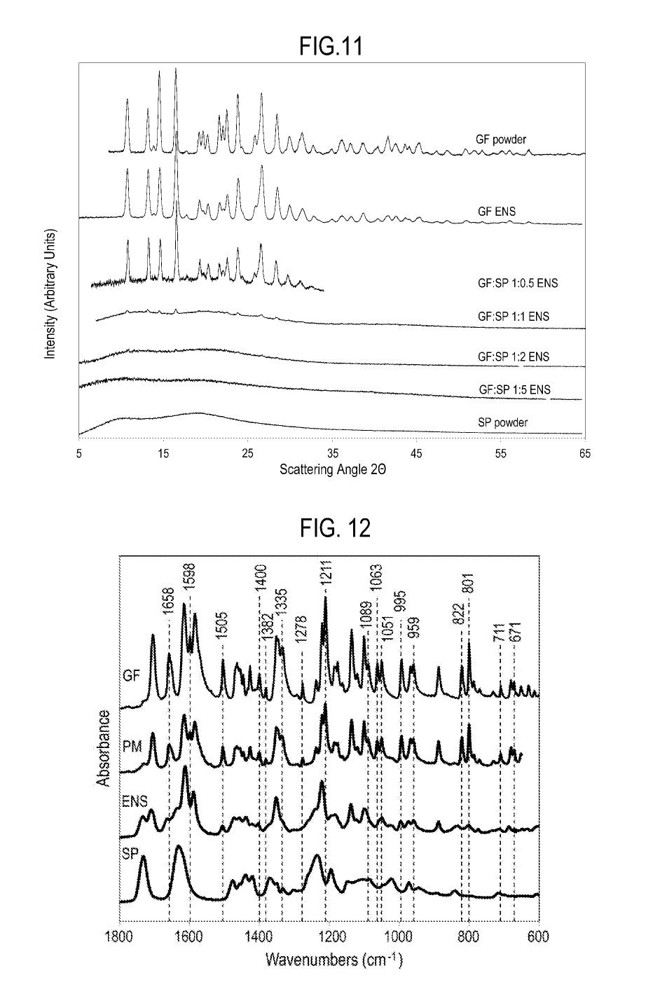

Dry powder produced as describe above in Examples 8-11 was evaluated in the biorelevant medium FaSSIF, comparing ENS-processed powders with physical mixtures at similar ratios of GF:SP. The results are summarized in FIG. 9. In pH 6.8 FaSSIF, GF dissolution profiles were similar for GF powder, GF:ENS and GF:SP physical mixtures (Controls 1, 3 and 5, respectively) and approximately double the equilibrium solubility previously measured for GF in water, which was from 6 mg/L to 10 mg/L. The curve for GF:SP-1:0.5-ENS (Example 8) was modestly higher than the control GF powders' solubility baseline. In contrast, values for Examples 9-11 (GF:SP-1:1, -1:2 and -1:5-ENS) rose quickly and the supersaturated concentrations persisted during the 5 hour experiment. Cₘₐₓ values were from 7- to 10-fold higher than GF powder (Control 1) (Table 2). Of these, the maximum concentration of 185 3 μg/mL was reached for Example 10 (GF:SP-1:2-ENS) at 30 min. The highest sustained levels were seen for Example 11 (GF:SP-1:5-ENS), which had a slightly lower Cₘₐₓ but showed sustained GF concentrations throughout the experiment, resulting in an area under the concentration-time curve (AUC) that was over 7-fold higher than measured for GF powder. Based on these results, it is likely that GF was maintained at supersaturated concentrations in the Caco-2 monolayer apical wells when SP was present in the test formulation.

TABLE 3

| | Control 1 | Example 8 | Example 9 | Example 10 | Example 11 | Control 9 | Control 10 |
|---|---|---|---|---|---|---|---|
| | GF | GF:SP- | GF:SP- | GF:SP- | GF:SP- | GF:SP- | GF:SP- |
| | powder | 1:0.5-ENS | 1:1-ENS | 1:2-ENS | 1:5-ENS | 1:2-PM | 1:5-PM |
| Tₘₐₓ (min) | 30 | 5.0 | 5.0 | 15 | 30 | 300 | 60 |
| Cₘₐₓ (μg/mL) | 18.3 | 33.7 | 163.6 | 185.3 | 136.8 | 18.6 | 21.6 |
| Ratio to Cₘₐₓ | — | 1.8 | 8.9 | 10.1 | 7.5 | 1.0 | 1.2 |
| for GF powder | | | | | | | |
| AUC | 5,202 | 7,890 | 24,527 | 35,265 | 38,330 | 5,252 | 6,039 |
| Ratio to AUC | — | 1.5 | 4.7 | 6.8 | 7.4 | 1.0 | 1.2 |
| for GF powder | | | | | | | |

### Impact of ENS-Processing on Physical State of the Powders

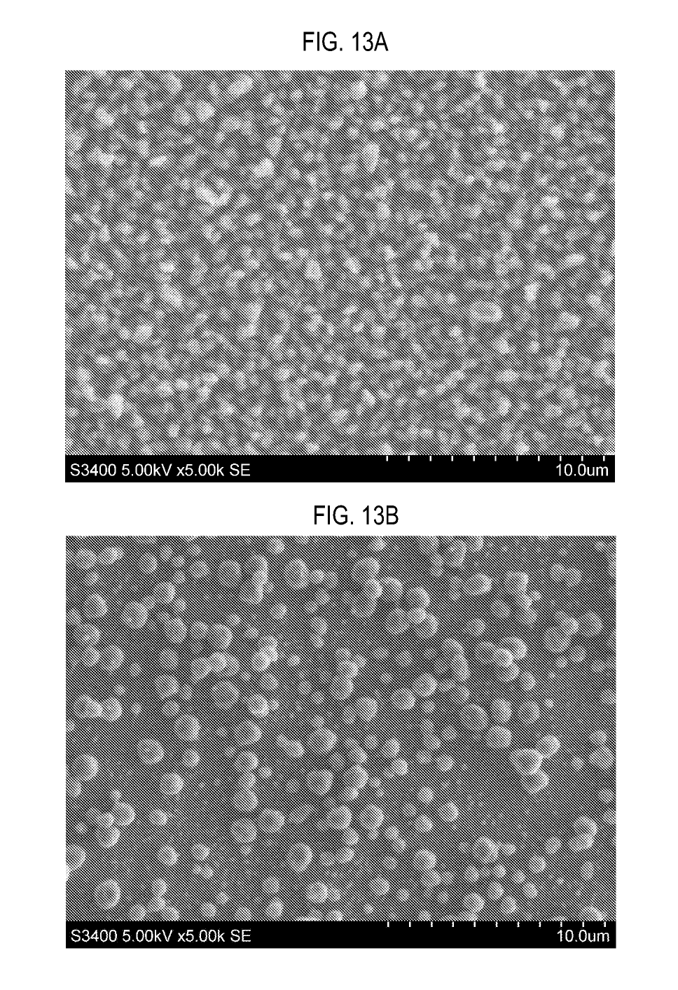

The amorphous properties of the ENS-processed GF:SP were confirmed by thermal analysis using differential scanning calorimetry (DSC). FIG. 10A shows a prominent crystal formation peak for ENS-produced material. A second tracing compares Example 9 (GF:SP-1:1-ENS) to a physical mixture (Control 5) and to GF powder (Control 1), neither of which exhibit recrystallization, as shown in FIG. 10B. The calculated levels of crystallinity based on DSC were 4% for (Example 9) GF:SP-1:1-ENS, 40% for (Control 6) GF:SP-1:1-SE, the solid produced by solvent evaporation, and 100% for (Control 3) (ENS-processed GF). Subsequent studies with Example 10 (GF:SP-1:2) and Example 12 (GF:SP-1:4-ENS) showed a complete conversion to the amorphous form while physical mixtures at those ratios were 100% crystalline.

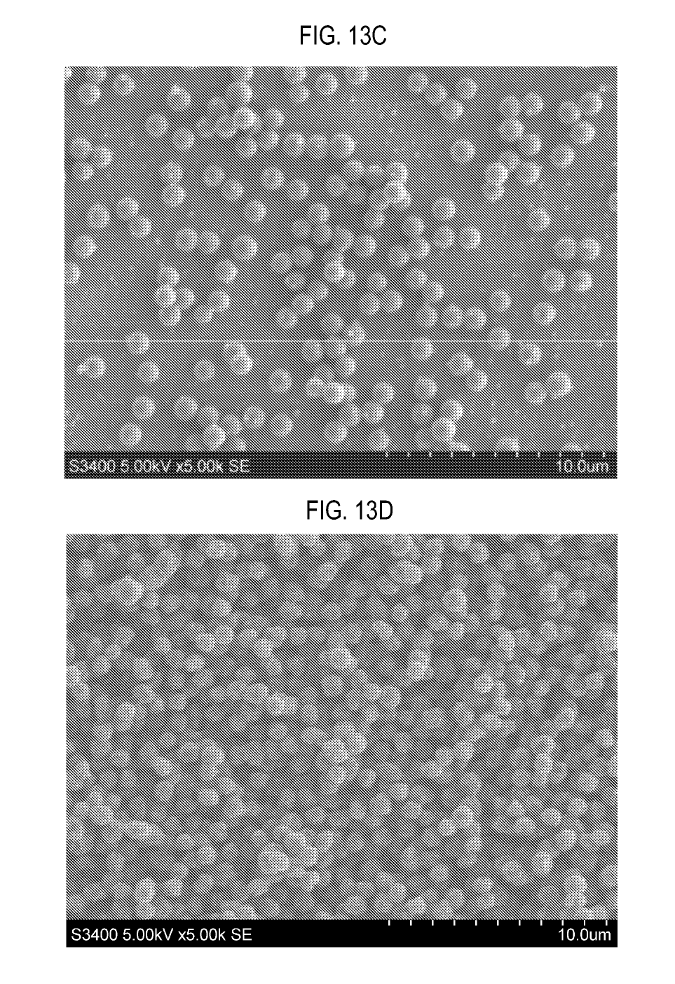

X-ray diffraction (XRD) was performed on the starting materials GF and SP. The diffraction plots for GF powder (Control 1) and GF-ENS (Control 3) were both consistent with completely crystalline material (FIG. 11). The plot for Example 8 (GF:SP-1:0.5-ENS) powder showed smaller but identifiable peaks and was estimated to be 42% crystalline. Example 9 (GF:SP-1:1-ENS) showed small residual peaks on an amorphous baseline and was estimated to be approximately 6% crystalline, consistent with the DSC findings. Residual peaks disappeared in the spectra for Example 10 (GF:SP-1:2) and Example 11 (GF:SP-1:5), estimated to be 2% and 0% crystalline, respectively. These were similar to the plot for SP alone (Control 4), which demonstrated its completely amorphous state. Both the DSC and XRD results indicate ENS-processed GF in the presence of adequate amounts of SP is primarily amorphous; that ENS processing of GF alone yields a crystalline material; and that physical mixing of GF and SP is insufficient convert the GF into an amorphous state.

TGA analysis showed weight loss in ENS-processed samples ranged from 2.10±0.01% for GF-ENS to 3.65±0.45% for GF:SP-1:2-ENS. To determine whether this reflected water or solvent evaporation, samples were analyzed by the Karl Fischer method, which showed that the water content of GF: SP-1:2-ENS was 5.0%±0.5%. Water content of the starting materials was 4.6% for SP and 3.8% for GF (replicate measurements were identical for both). Water content of the solvent spray solution for GF:SP-1:2-ENS was 2.3%. This suggests that the primary residual in the ENS-processed powders was water rather than solvent.

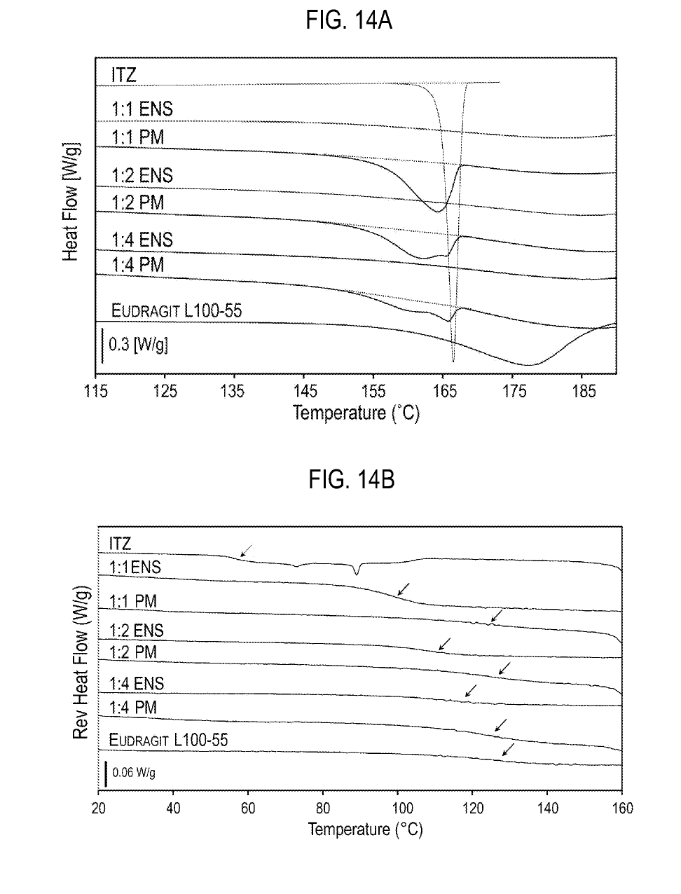

FTIR spectra were used to analyze the nature of the molecular interaction between GF (Control 1) and SP (Control 4) in the dry powders and how the molecular interactions might vary with the processing method. Comparison of FTIR spectra for GF:SP-1:1-ENS (Example 9) to that for GF:SP-1:1-PM (Control 5) (FIG. 12) shows a major difference for the absorbance band at 1658 cm⁻¹, corresponding to stretching of the carbonyl group of cyclohexene ring. GF:SP-1:1-ENS (Example 9) exhibits a marked decrease in stretching intensity compared to GF:SP-1:1-PM (Control 5). A peak at 1598 cm⁻¹ that is present for both GF powder (Control 1) and GF:SP-1:1-PM (Control 5) physical mix, corresponding to stretching of the conjugated double bond —C═C—C═O, has disappeared in the spectrum for GF:SP:1:1-ENS (Example 9). The stretching band at 1505 cm⁻¹, which is characteristic of aromatic ring vibrations, and the absorption band at 1335 cm⁻¹ both diminished or disappeared in GF:SP:1:1-ENS (Example 9). The absorption bands that appear in the fingerprint region below 1500 cm⁻¹ also showed marked differences for GF:SP-1:1-ENS (Example 9) and GF:SP1:1-PM (Control 5). These results suggest intermolecular interactions in the ENS-produced powder that are not observed in the physical mix.

Without being bound by theory, the present inventors believe the amorphous state and the submicron particle size of the ENS-processed GF and SP formulations each play a role in the speed and degree of supersaturation observed in the dissolution experiments. Without being bound by theory, the present inventors believe the mechanism by which SP inhibits GF precipitation is likely due to the surfactant-like properties contributed by its polymeric components and its ability to form micelles rapidly in solution. A higher amount of SP in the formulation, relative to GF, increases the duration of the inhibitory effect, as is seen in comparison of the GF:SP powders of 1:5 (Example 7) versus 1:2 (Examples 6) and 1:1 (Examples 5) powders. In contrast, an equivalent physical mixture of the polymer with the crystalline drug powder shows no increased dissolution beyond the equilibrium solubility of GF in FaSSIF. The particle size of GF:SP-1:0.5-ENS by SEM was similar to GF:SP formulations with higher SP content, but its apparent solubility increased only modestly relative to controls with no SP. Its crystallinity was 74% while the other formulations with higher SP were largely amorphous. Particle size alone was insufficient to drive similar levels of supersaturation.

### Controls A1-A8

Control A1 was ITZ.

Control A2 was SPORONOX.

Controls A3-A8 were prepared by mixing ITZ and polymer at room temperature in a ceramic mortar and pestle with gentle force for about 5 minutes or until a fine and uniform mixture was observed. E100 pellets were crushed before the itraconazole powder was added. The mixtures were then transferred to glass vials, capped, and stored in a desiccator at room temperature. The polymer and the ratio of ITZ to polymer were as follows: Control A3 L100-55 1:2, Control A4 L100-55 1:4, Control AS E100 1:2, Control A6 E100 1:4, Control A7 L100-55 1:1, and Control A8 E100 1:4

Control A9 was L100-55

Control A10 was E100

### Examples A1-A6

Spray solutions were prepared by first dissolving the polymer in a 90 mL blend of 3:2 v/v ethanol:acetone. ITZ was then dissolved in 10 mL of dichloromethane, and the resulting solution was added to the polymer solution to form a spray solution. The type of polymer and the ratio of ITZ and polymer in each spray solution on a weight to weight (w/w) basis were as follows: Example A1 L100-55 1:1, Example A2 L100-551:2, Example A3 L100-55 1:4, Example A4 E100 1:1, Example A5 E100 1:2, and Example A6 E100 1:4.

A 20 mL aliquot of each spray solution was filtered through a 0.45 μm polytetrafluoroethylene filter. The spray solutions were sprayed using the ENS System according to the ENS Process to form the dry powders of Examples A1-A6. A stable cone-jet mode electrospray was achieved at 29.8 kV. The dry powder was collected on the stainless steel plate, gently scraped off, and stored in a desiccator.

Examples A1-A6 and Controls A1-A8 were tested according to the Scanning Electron Microscopy, Differential Scanning calorimetry (DSC), Thermal Gravimetric Analysis, Fourier Transform Infrared Spectroscopy and Nuclear Magnetic Resonance Test Methods to determine the percent crystallinity and the presence of an amorphous composition, as indicated by the absence of the melting endotherm for crystalline ITZ at approximately 169° C. The results are reported below.

Scanning Electron Microscopy

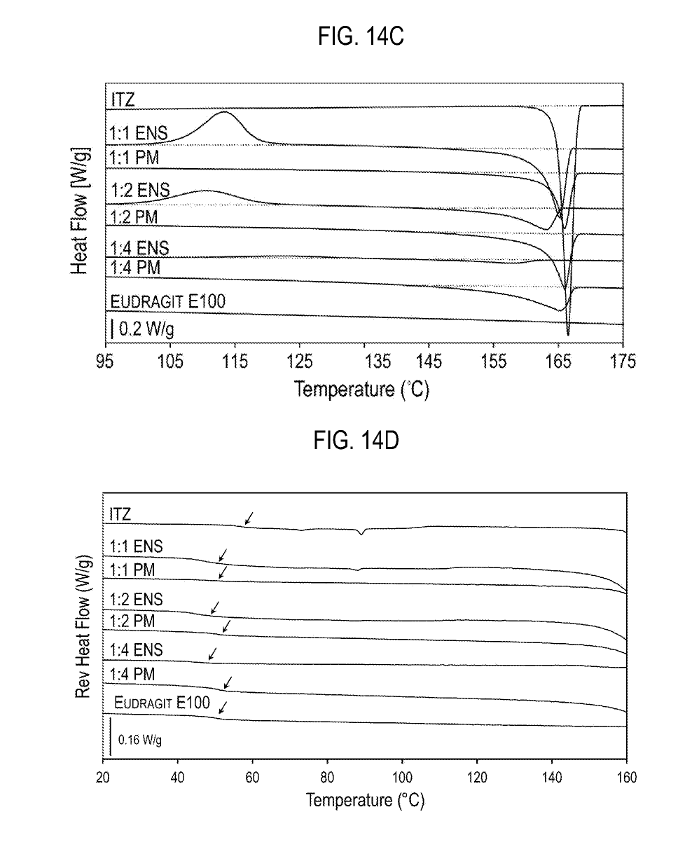

Scanning electron microscopy images of the following ENS-sprayed ITZ formulations: Example A2 1:2 ITZ:L100-55, Example A5 1:2 ITZ:E100, Control A1, ITZ and 1% K30 polyvinylpyrrolidone are shown, on a 10 μm scale, in FIGS. 13A-13D, respectively. Pure ITZ had an irregular crystalline shape whereas the ENS powder was spherical with smaller internal average diameter (800 nm). The uniform appearance of the spray particles suggests a close interaction of the polymer and drug, without obvious crystals or visible phase differences.

Differential Scanning Calorimetry Ollowing

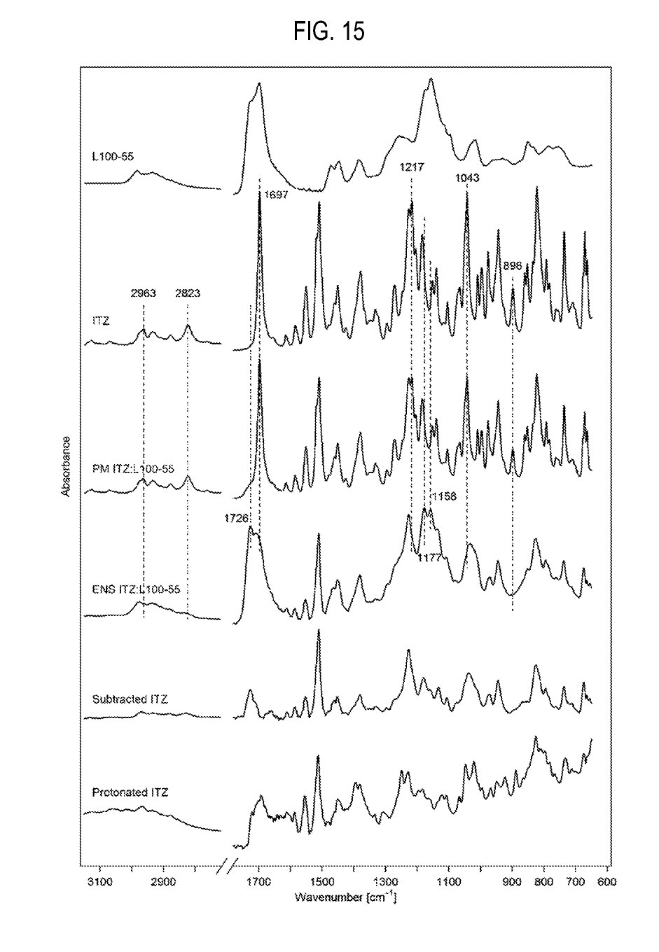

Controls and Examples were tested according to the DSC test method. The DSC thermograms of ITZ alone showed an endothermic peak at 169° C., corresponding to its melting point. The thermograms of ITZ:L100-55 and the corresponding physical mixtures show that ITZ was entirely amorphous for all EUDRAGIT L100-55 formulation ratios, as indicated by the absence of the 169° C. melting endotherm for crystalline ITZ; ITZ formulations with EUDRAGIT E100 at a ratio 1:4 showed complete conversion of a crystalline form of ITZ to an amorphous form of ITZ. The conversion of ITZ from a crystalline form to an amorphous form was progressive as the ratio of ITZ to EUDRAGIT E100 was increased from 1:1, 1:2 and 1:4. Complete conversion with EUDRAGIT L100-55 could be attributed to the chemical structure of the polymer having a carboxylic acid group providing a strong proton donor suitable for hydrogen bonding and enhancing the structural stabilization of the amorphous form by limiting its free movement within the polymer crystal lattice. The DSC data appeared to support the formation of a fully dispersed solid, as evidenced by a single Tg to form a single phase and no detectable crystallinity from either component formed during co-solidification (FIGS. 14A-14D).

FIGS. 14A and 14B are DSC thermograms of ITZ (Control A1) and EUDRAGIT L100-55 (Control A9), as physical mixtures (Controls A3, A4, and A7) and as formed using ENS at various ITZ:EUDRAGIT L100-55 ratios (Examples A1-A3). FIG. 14A is a heat flow DSC and FIG. 14B is a reversed heat flow DSC.

FIGS. 14C and 14D are DSC thermograms of ITZ (Control A1) and EUDRAGIT E100 (Control A10), as physical mixtures (Controls A5, A6, and A8), and as formed using ENS at various ITZ: EUDRAGIT E100 ratios (Examples A4-A6). FIG. 14C is a heat flow DSC and FIG. 14D is a reversed heat flow DSC.

Thermal Gravimetric Analysis

Thermal analysis showed a weight change of less than 0.1%, suggesting minimal residual solvent was present in dry 1:2 ITZ:EUDRAGIT L100-55 and ITZ:EUDRAGIT E100 powders.

Fourier Transform Infrared Spectroscopy

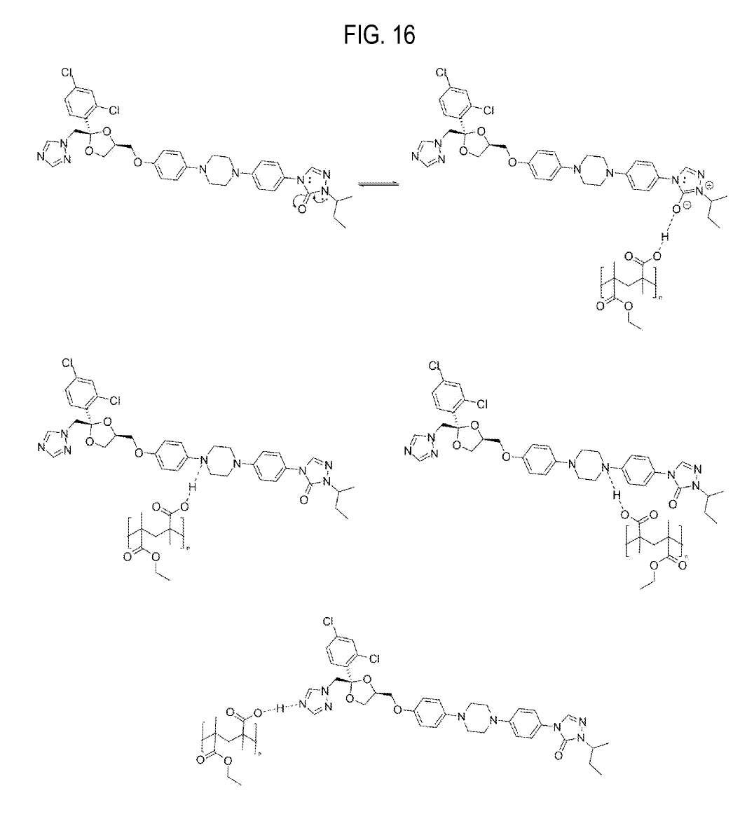

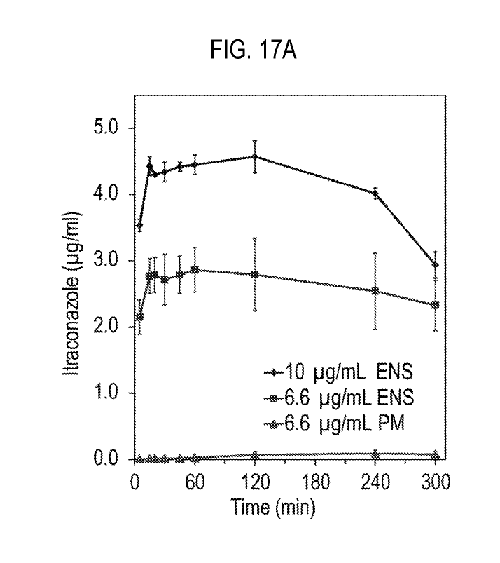

FTIR Infrared spectra from 4000 to 650 cm⁻¹ of ITZ, EUDRAGIT L100-55, physical mixtures of ITZ and polymer, ENS-sprayed ITZ powder, and ITZ treated with 35% hydrochloric acid (i.e., protonated ITZ) were obtained and recorded. The FTIR spectrum of ITZ showed characteristic peaks at 600-1800 cm-1 corresponding to stretching and vibrations of functional groups —C═C—, aromatic rings, aromatic —Cl, and a strong peak observed at 1690 cm⁻¹ attributed to the stretching of the carbonyl group of 1,2,4-triazolin-5-one, whereas C—H peaks stretching for aromatic and alkanes groups were observed at the 2800-3200 cm⁻¹ region (FIG. 15); EUDRAGIT L100-55 showed characteristic peaks at 600-1800 cm⁻¹ corresponding to C—H bending, C—O stretching, and a split peak at 1698 cm⁻¹ corresponding to stretching of the carbonyl of the ester and the carboxylic acid.

FTIR spectra of the physical mixture and ITZ showed close similarity across all regions. Comparison of FTIR spectra of ENS powder to physical mixture show substantial differences in the 2500-3500 cm⁻¹ and 600-1800 cm⁻¹ regions. The ENS powder spectra showed a split peak and a downshift maxima at 1726 and 1709 cm⁻¹, which are the result of hydrogen bonding between the carboxylic acid of the polymer and various sites as shown in FIG. 15. Subtraction of EUDRAGIT L100-55 spectra from ENS spectra results in spectra similar to ITZ protonated with hydrochloric acid, especially in the lower 600-1800 cm⁻¹ region and most noticeably the carbonyl stretching region.

FIG. 15 includes Fourier transform infrared spectra showing the infrared absorption of EUDRAGIT L100-55, itraconazole, a physical mixture of ITZ and EUDRAGIT L100-55 at a ratio of 1:1, ENS powder of ITZ and EUDRAGIT L100-55 at a ratio of 1:1, ITZ protonated with HCl, and subtracted profile of ENS-EUDRAGIT L100-55.

### Dissolution in Fasted State Simulated Intestinal Fluid Medium

Example A1 was tested according to the Dissolution In Biorelevant Medium Using Non-Sink Condition Test Method with the following exceptions. Amounts of Example A1, i.e., 1:1 ITZ: EUDRAGIT L100-55 powder, sufficient to achieve concentrations of approximately 6.6 μg/mL and 10 μg/mL ITZ, was added, in duplicate, to separate flasks. The amounts of 6.6 μg/mL and 10 μg/mL ITZ are the equivalent to about 80 and 125 times, respectively, the solubility of ITZ in FaSSIF medium, which was experimentally determined to be 80 ng/mL.

Control A7, which consisted of a physical mixture of ITZ and L100-55 at a ratio of 1:1, was added to another flask, in an amount sufficient to achieve a concentration of 6.6 μg ITZ/mL dissolution media.

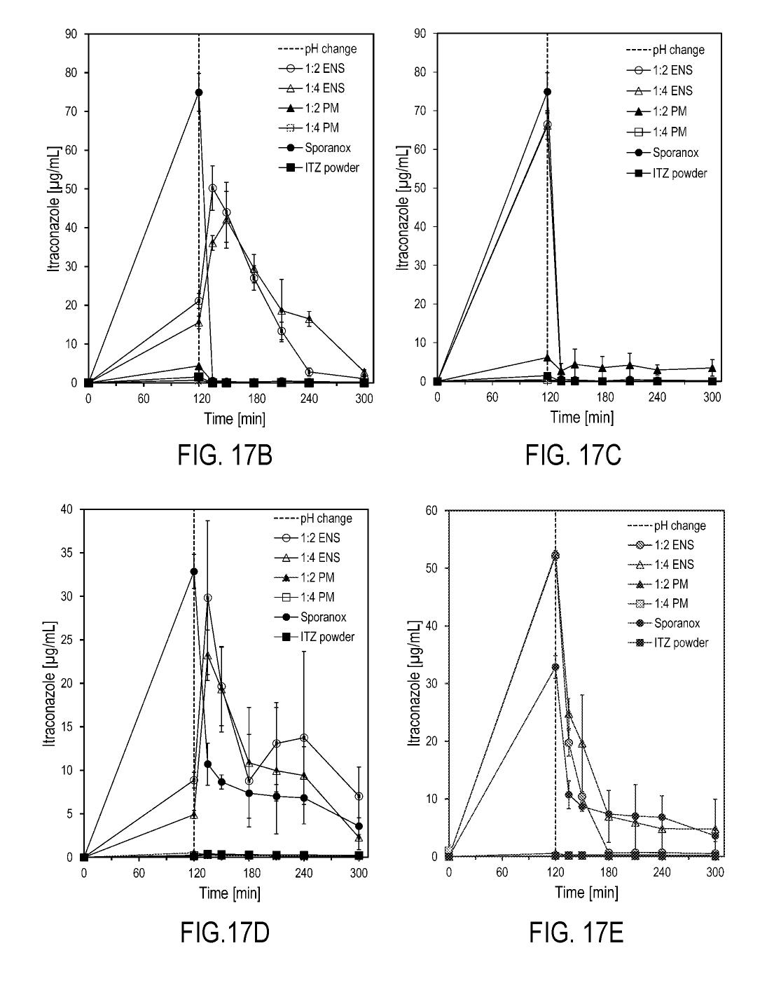

The filtered samples were analyzed for ITZ concentration using an HPLC system according to the aforementioned test method with the following exceptions: the HPLC system was coupled to a photodiode array detector connected in series with a mass selective detector operating in electrospray positive mode and a selective ion monitoring mode for m/z 706.5 corresponding to [M+H]⁺ for confirmation; the injection volume was 10 μL and quantification was performed using 263 nm λₘₐₓ the mobile phase was isocratic at 0.3 mL/min and consisted of 0.1% formic acid in water and 0.1% formic acid in acetonitrile in a 20:80 ratio, the UV calibration curve was linear ranging from 0.016 μg/mL to 50 μg/mL, and quantification was performed based on linear peak response factor ratio. The resulting dissolution profiles of Examples A1 and Control A7 are plotted in FIG. 17A.

When Examples A2 and A3 are tested according to the Dissolution In Biorelevant Medium Test Method they are expected to have similar dissolution profiles as Example A1 and are expected to outperform formulation 1:1, i.e., Example A1. The same observation is expected to be found for formulations that include ITZ and EUDRAGIT E100, i.e., Examples A4-A6 with formulation 1:4 expected to outperform 1:2.

Dissolution in Buffer Media with pH Transition

Examples A2, A3, A5 and A6 and Controls A1-A6 were tested according to the Dissolution in Buffer Media with pH Transition Test method. Dissolution vessels were filled with an acidic medium that consisted of 75 mL 0.1 N HCl and had a pH 1. Itraconazole, in an amount of 6 mg, which is equivalent to about 20 times its assumed solubility of 4 μg/mL in 0.1 N HCl, was added to the acidic medium. The solution was left stirring for 120 minutes, after which 3 mL aliquots were drawn from each vessel. Immediately after the aliquots were removed from the vessels, 25 mL of 0.2 M tribasic sodium phosphate buffer was added to each vessel to produce in a dissolution media having a pH 6.8±0.05. Subsequently, 3 mL aliquots were drawn from each vessel after 135 min, 150 min, 180 min, 210 min, 240 min, and 300 min. The aliquots were then analyzed to determine the concentration of itraconazole present in the media. The results were recorded in μg/mL.

The concentration at 120 minutes (C₁₂₀), the maximum concentration (Cmax), the time at which the maximum concentration was reached (Tmax), and the area under the curve (AUC), obtained from the dissolution test results are summarized in Table A1. Since only one sample was collected before the pH transition, the AUC, Tmax and Cmax were calculated based on the results following pH transition.

The dissolution profiles for Examples A2 and A3 and Controls A1-A4 are and plotted in FIG. 17B. The dissolution profiles for Examples A5 and A6, and Controls A1, A2 and A5-A6 are plotted in FIG. 17C.

TABLE A1

| | | Acid | | | | | | |
|---|---|---|---|---|---|---|---|---|
| | | Phase | Neutral Phase | | | | | |
| | | C₁₂₀ | Cₘₐₓ | Tₘₐₓ | AUC (SD) | AUC Ratio | | |
| Example | | (μg/mL) | (μg/mL) | (hrs) | (μg · hr/mL) | ENS/PM | ENS/ITZ | ENS/SPO |
| Control A1 | ITZ | 1.50 | 0.061 | 3.50 | 0.218 (0.078) | — | — | — |
| | Powder | | | | | | | |
| Control A2 | SPO | 74.9 | 0.578 | 2.25 | 10.3 (0.992) | — | — | — |
| | Beads | | | | | | | |
| Example A2 | 1:2 | 21.1 | 50.2 | 2.25 | 54.5 (7.73) | 40.8 | 250 | 5.32 |
| | ITZ:L100- | | | | | | | |
| | 55 (ENS) | | | | | | | |
| Control A3 | 1:2 | 4.35 | 0.388 | 3.50 | 1.34 (0.049) | | | |
| | ITZ:L100- | | | | | | | |
| | 55 (PM) | | | | | | | |
| Example A3 | 1:4 | 15.6 | 42.0 | 2.50 | 64.5 (3.56) | 760 | 295 | 6.29 |
| | ITZ:L100- | | | | | | | |
| | 55 (ENS) | | | | | | | |
| Control A4 | 1:4 | 0.679 | 0.00 | 2.25 | 0.085 (0.006) | | | |
| | ITZ:L100- | | | | | | | |
| | 55 (PM) | | | | | | | |
| Example A5 | 1:2 | 66.5 | 0.00 | 2.25 | 8.31 (0.351) | 0.764 | 38.0 | 0.810 |
| | ITZ:E100 | | | | | | | |
| | (ENS) | | | | | | | |
| Control A5 | 1:2 | 6.15 | 4.41 | 2.50 | 10.90 (6.37) | | | |
| | ITZ:E100 | | | | | | | |
| | (PM) | | | | | | | |
| Example A6 | 1:4 | 66.1 | 0.00 | 2.25 | 8.26 (0.439) | 4.995 | 37.8 | 0.806 |
| | ITZ:E100 | | | | | | | |
| | (ENS) | | | | | | | |
| Control A6 | 1:4 | 8.35 | 1.42 | 2.25 | 1.66 (0.150) | | | |
| | ITZ:E100 | | | | | | | |
| | (PM) | | | | | | | |

EUDRAGIT L100-55 showed an approximately 3-fold increase in concentration following the pH transition and maintained supersaturated concentrations for over 3 hours. The AUC was about five- to six-fold higher than the AUC of SPO, i.e., Control A2. The pH transition for the examples that included EUDRAGIT E100, i.e., Examples

A5 and A6, resulted in flocculation and an immediate drop of concentration to an undetectable level. Similar drops in itraconazole concentration following pH transition were observed for Controls A2 (SPO), A5 1:2 (ITZ:E100) and A6 1:4 (ITZ:E100).

Dissolution in Biorelevant Media with pH Transition

Examples A2, A3, A5 and A6 and Controls A1-A4 were tested according to the Dissolution in Biorelevant Media with pH Transition Test Method. The method included first pacing the samples in an acid stage and then converting the acid stage to a neutral stage. The acid stage consisting of 75 mL of pH 1.5 FaSSGF prepared by dissolving 0.469 g sodium chloride, 0.395 g sodium phosphate monobasic monohydrate, 0.120 g sodium hydroxide, and 0.220 g SIF powder in 25 mL HPLC water. The pH of this solution was adjusted to 10.30 with 1N NaOH solution. Then 25 mL of this solution was added to the acidic medium in the vessel such that the resulting composition was similar in composition to FaSSIF biorelevant medium having a pH 6.5±0.05.

Dissolution testing was performed in triplicate for Controls A1-A4 and Examples A2, A3, A5 and A6. The shell of each SPO capsule containing 100 mg itraconazole was gently broken and the contents weighed on analytical balance from which the equivalent of 6 mg of itraconazole was used for each dissolution sample.

The concentration at 120 minutes (C₁₂₀), the maximum concentration (Cmax), the time at which the maximum concentration was reached (Tmax), and the area under the curve (AUC), obtained from the dissolution test results are summarized in Table A2. Since only one sample was collected before the pH transition, the AUC, Tmax and Cmax were calculated based on the results following pH transition.

The dissolution profiles for Examples A2 and A3 and Controls A1-A4 are and plotted in FIG. 17D. The dissolution profiles for Examples A5 and A6, and Controls A1, A2 and A5-A6 are plotted in FIG. 17E.

TABLE A2

| | | Acid | | | | | | |
|---|---|---|---|---|---|---|---|---|
| | | Phase | Neutral Phase | | | | | |
| | | C₁₂₀ | Cₘₐₓ | Tₘₐₓ | AUC (SD) | AUC Ratios | | |
| Sample | | (μg/mL) | (μg/mL) | (hrs) | (μg · hr/mL) | ENS/PM | ENS/ITZ | ENS/SPO |
| Control A1 | ITZ | 0.112 | 0.291 | 2.25 | 0.75 (0.221) | — | — | — |
| | Powder | | | | | | | |
| Control A2 | Sporanox | 32.9 | 10.7 | 2.25 | 24.1 (1.17) | — | — | — |
| | Beads | | | | | | | |
| Example A2 | 1:2 | 8.86 | 29.8 | 2.25 | 40.7 (16.8) | 73.6 | 54.2 | 1.69 |
| | ITZ:L100- | | | | | | | |
| | 55 (ENS) | | | | | | | |
| Control A3 | 1:2 | 0.521 | 0.277 | 2.25 | 0.553 (0.012) | | | |
| | ITZ:L100- | | | | | | | |
| | 55 (PM) | | | | | | | |
| Example A3 | 1:4 | 4.90 | 23.2 | 2.25 | 32.2 (3.89) | 38.0 | 43.0 | 1.34 |
| | ITZ:L100- | | | | | | | |
| | 55 (ENS) | | | | | | | |
| Control A4 | 1:4 | 0.392 | 0.392 | 2.25 | 0.848 (0.114) | | | |
| | ITZ:L100- | | | | | | | |
| | 55 (PM) | | | | | | | |
| Example A5 | 1:2 | 52.2 | 19.7 | 2.25 | 16.8 (0.337) | 43 | 22.4 | 0.70 |
| | ITZ:E100 | | | | | | | |
| | (ENS) | | | | | | | |
| Control A5 | 1:2 | 0.55 | 0.188 | 2.25 | 0.39 (0.027) | | | |
| | ITZ:E100 | | | | | | | |
| | (PM) | | | | | | | |
| Example A6 | 1:4 | 52.5 | 24.8 | 2.25 | 23.4 (2.00) | 118 | 31.1 | 0.97 |
| | ITZ:E100 | | | | | | | |
| | (ENS)* | | | | | | | |
| Control A6 | 1:4 | 0.428 | 0.125 | 2.5 | 0.198 (0.086) | | | |
| | ITZ:E100 | | | | | | | |
| | (PM) | | | | | | | |

*The data are based on n = 2 due to technical problems encountered with one of the triplicate dissolution vials.
ENS = produced by ENS spray process
PM = physical mixture

Examples A2 and A3 (i.e., ITZ:L100-55 1:2 and 1:4 formulations) showed a 4- to 5-fold jump in concentration following the pH transition. A remarkable drop in concentration for Control A2 (SPO) was observed but unlike the results in the buffer system, concentrations remained at supersaturation levels, even though Control A2 (SPO) was outperformed by both ITZ: EUDRAGIT L100-55 formulations (Examples A2 and A3) with Example A2 (i.e., the 1:2 ITZ:EUDRAGIT L100-55 formulation) outperforming Example A3 (the 1:4 ITZ:EUDRAGIT L100-55 formulation).

The concentration of itraconazole from Examples A5 and A6 (1:2 and 1:4 ITZ:E100) was about 1.5-times higher than Control A2 (SPO) in the FaSSGF but slightly lower in acidic buffer. Following the pH transition, itraconazole concentrations for both dropped, similar to levels seen for SPO, but only concentrations for Example A6 (i.e., the 1:4 ITZ:E100 formulation) remained at similar supersaturation levels. Flocculation was observed for both EUDRAGIT E100 formulations (Examples A5 and A6) and SPO (Control A2) following the pH transition, which is consistent with the fall in Itraconazole concentrations. The flocculants were filtered off and dried in vacuum at room temperature. HPLC analysis showed that itraconazole was entrapped within the flocculants and DSC confirmed that the flocculant entities were amorphous.

### Control A7 and Examples A7 and A8

The spray solutions of Control A7 and Examples A7-A8 were prepared as follows. Control A11 ITZ was dissolved in dichloromethane to form a solution having a concentration of ITZ of 1% w/v. Examples A7 E100 polymer was dissolved in a (3:2 v/v) blend of ethanol and acetone to form a polymer solution. ITZ was dissolved in dichloromethane and then added to the polymer solution, resulting in a solution having a concentration of ITZ of 1% w/v and E100 of 2% w/v. Examples A8 were prepared according to Example A7 with the exception that the polymer was L100-55.

The spray solutions of Control A7 and Examples A7 and A8 were then sprayed according to the ENS Process onto a stainless steel plate located 15.25 cm beneath the nozzle array to form a dry powder. The powder was harvested from the plate and stored in desiccant until characterization and dissolution testing. Examples A7 and A8 were stored at room temperature for 20 weeks and ambient humidity prior to testing using XRD.

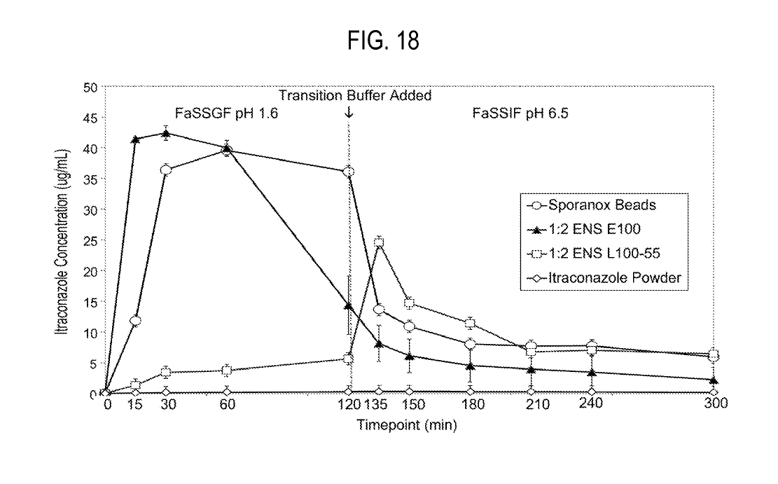

The ENS powders of Examples A7 and A8 and Controls A1 and A2 were tested according to the Dissolution in Biorelevant Media with pH Transition Test Method (n=3 per formulation) using 100 mL aliquots of FaSSGF, which was transitioned to FaSSIF after two hours using Transition Medium. The dissolution profiles of Examples A7 and A8 and Controls A1 and A2 are plotted in FIG. 18.

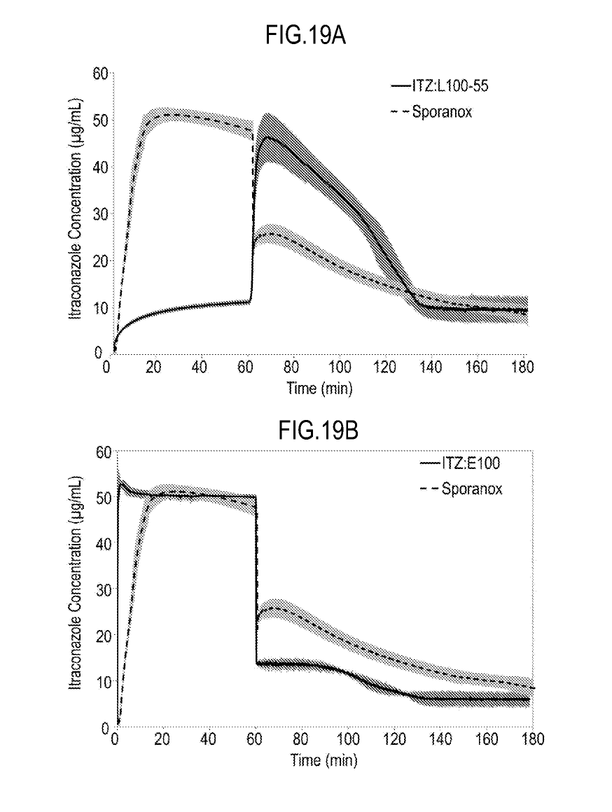

Examples A7 and A8 were then analyzed using real-time monitoring using a μDISS Profiler fiber optic ultraviolet spectroscopy system (pION Instruments, Billerica, Mass.). Test vessels were filled with 15 mL FaSSGF. The medium in each vessel was maintained at 37° C. throughout the analysis. The powder of Control A2 and Examples A7 and A8, in an amount sufficient to provide 1.2 mg itraconazole equivalent per vessel, was added to separate vessels. The itraconazole concentration was measured for 1 hour, then 5 mL of the Transition Medium was added to the vessel, causing the medium to transition to a FaSSIF medium having a pH of 6.5. The itraconazole concentration in each vessel was then measured for an additional 2 hours. The results are plotted in FIGS. 19A and 19B.

### Particle Size Analysis

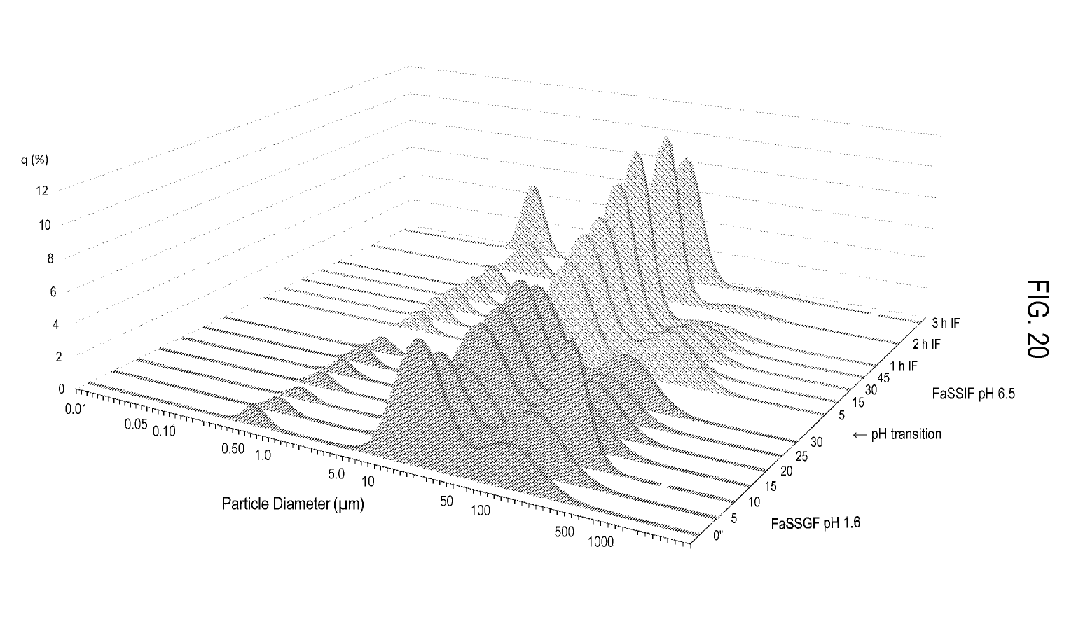

Control A7 and Examples A7 and A8 were studied using laser diffraction. The particle size of the powders of Control A7 and Examples A7 and A8 was measured throughout the Dissolution with pH Transition Test Method using a Horiba LA-960 laser diffraction analyzer fitted with a 10 mL sample vessel. Repeated measurements were made during the first 30 min while the medium was FaSSGF, and then over a 3 hour period after the medium was transitioned to FaSSIF. The results are plotted in FIGS. 20, 21, and 22, for Control A7, Example A8 and Example A7, respectively.

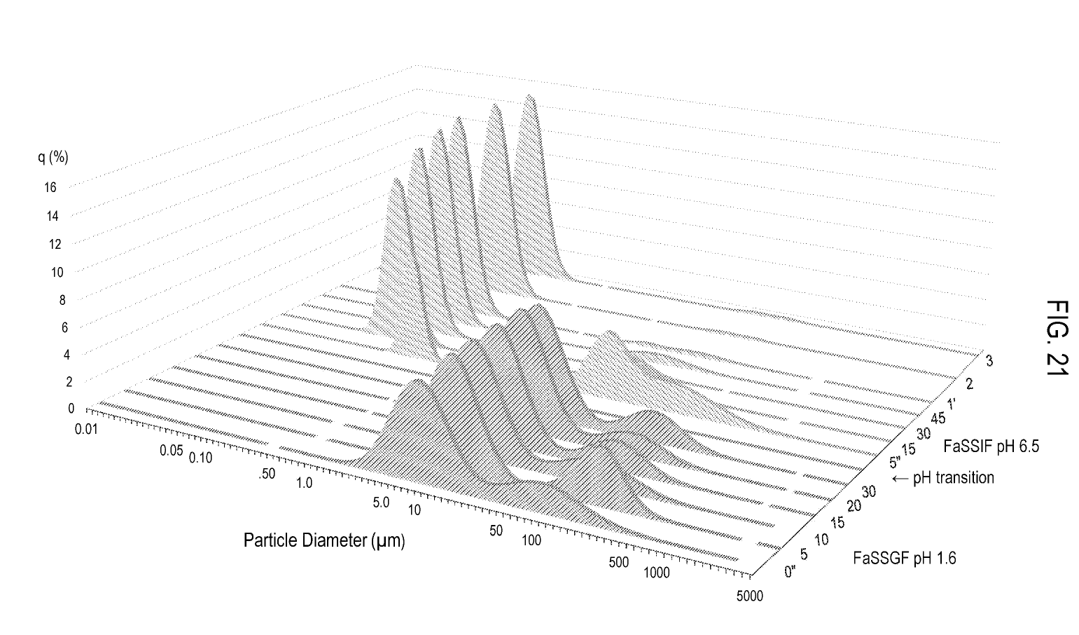

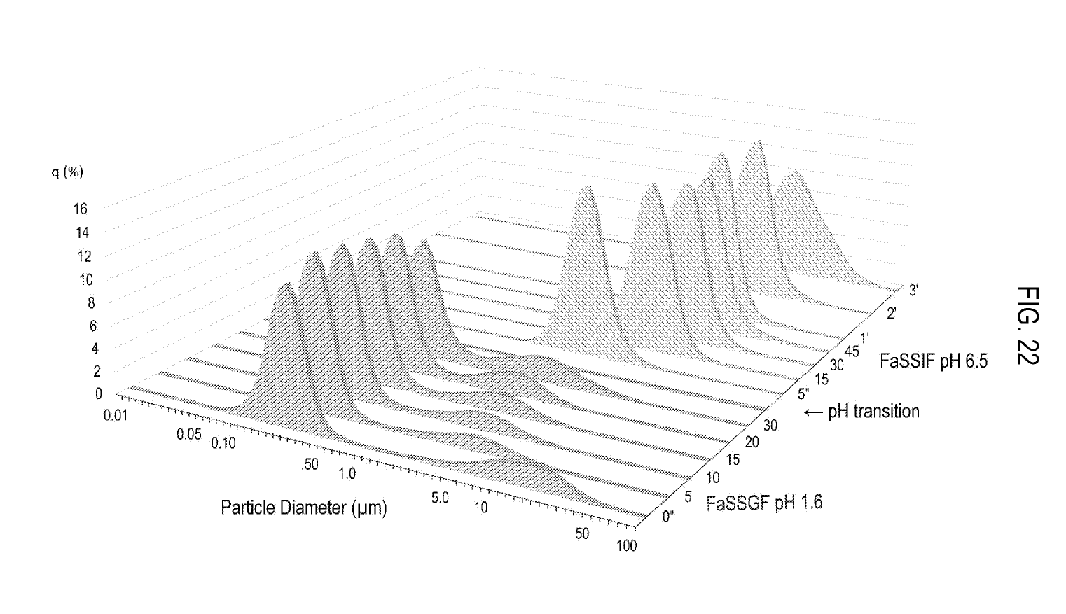

The size of the particles of Control A7 was relatively larger than the size of the particles of Example A8. When the pH of the dissolution media was increased from 1.6 to 6.5, the size of the particles of Control A7 did not change, as illustrated by the plot in FIG. 20. The size of the particles of Example A8 became smaller when the pH was increased from 1.6 to 6.5, as illustrated in the plot in FIG. 21. The size of the particles of Example A7 became larger when the pH was increased from 1.6 to 6.5, as illustrated by the plot in FIG. 22.

### XRD Analysis

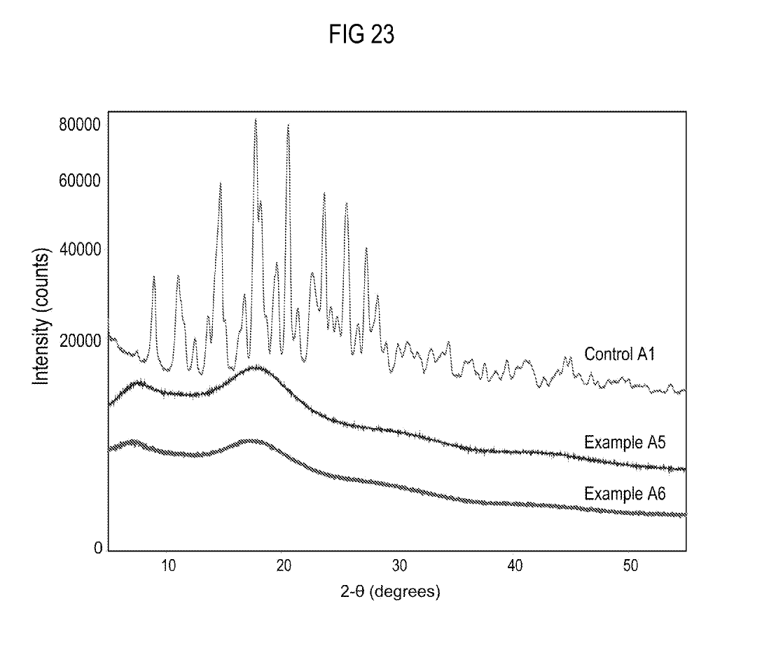

The XRD spectra of Control A1 and Examples A7 and A8 are shown, from top to bottom, in FIG. 23.

### Example B

A series of crystalline drug and polymer combinations were dissolved in solvents in the amounts and at the ratios set forth in Table B. The solutions were then sprayed using the ENS system described above. The resulting dry compounds were collected and analyzed according to the XRD, DSC and SEM test methods. The results are reported in Table B.

TABLE B

| | | | Concentration | | |
|---|---|---|---|---|---|
| | | | of Compound | | Description of SEM images of ENS- |
| | Polymer | | in Spray | Ratio of | processed powders, with particle size ranges. |
| | and | | Solution | Compound | XRD and DSC determinations of physical |
| Compound | Excipients | Solvent System | (mg/mL) | to Polymer | state (amorphous or crystalline). |
| Triamcinolone | None | EtOH:Ace | 10 | N/A | Spheroidal, uniformly porous 1.0 to 1.2 μm |
| Acetonide | | | | | particles. XRD not tested. DSC consistent |
| | | | | | with partially amorphous material. |
| Triamcinolone | Tween 80 | EtOH:Ace | 10 | 100:1 | Spheroidal, uniformly porous 0.6 to 1.0 μm |
| Acetonide | | | | | particles. XRD not tested. DSC consistent |
| | | | | | with largely amorphous material. |
| Asenapine | None | EtOH:Ace | 10 | N/A | Smooth spheroidal ~0.8-1.0 μm particles |
| Maleate | | | | | with fused boundaries, multiple satellite |
| | | | | | particles. XRD not tested, DSC consistent |
| | | | | | with largely amorphous material. |
| Asenapine | 30 | EtOH:Ace | 10 | 1:2 | Discrete, dimpled, spheroidal, smooth ~0.8 |
| Maleate | | | | | to 1.0 μm particles with some satellite |
| | | | | | particles. DSC consistent with largely |
| | | | | | amorphous material. |
| Budesonide | None | EtOH:Ace | 10 | N/A | Smooth spheroidal ~1.0 μm particles with |
| | | | | | boundaries fused with adjacent particles, |
| | | | | | interspersed with multiple satellite particles |
| | | | | | ~200 nm or less. XRD and DSC not tested. |
| Budesonide | L100-55 | EtOH:Ace | 10 | 1:2 | A population of larger 1.0-2.0 μm collapsed |
| | | | | | spheroidal particles among smaller ~100-200 nm |
| | | | | | irregular, teardrop-shaped particles. |
| | | | | | XRD and DSC not tested. |
| Budesonide | S100 | EtOH:Ace | 10 | 1:2 | Discrete, collapsed spheroidal particles |
| | | | | | ranging from 400 to 600 nm, some teardrop- |
| | | | | | shaped. XRD and DSC not tested. |
| Naproxen | None | EtOH:Ace | 10 | N/A | No discrete particles, rough-edged fused |
| | | | | | particulate remnants with possible crystalline |
| | | | | | elements, <1.0 μm in transverse diameter. |
| | | | | | DSC and XRD consistent with largely |
| | | | | | crystalline material. |
| Naproxen | E100 | EtOH:Ace | 10 | 1:1 | Discrete, spheroidal, 0.4 to 1.0 μm particles. |
| | | | | | DSC and XRD consistent with completely |
| | | | | | amorphous material. |
| Naproxen | HPMC- | EtOH:Ace | 10 | 1:1 | Fused particulate elements with no discrete |
| | AS | | | | particles. XRD and DSC consistent with |
| | | | | | partially crystalline material. |
| Indomethacin | None | EtOH:Ace | 10 | N/A | Extensive interparticle bridging of smooth |
| | | | | | spheroidal, submicron particle remnants, no |
| | | | | | discrete particles. DSC and XRD consistent |
| | | | | | with mainly amorphous material. |
| Indomethacin | K30 | EtOH:Ace | 10 | 1:1 | Collapsed and discoid smooth particles ~1.0 μm |
| | | | | | in diameter, many covered with satellite |
| | | | | | particles. XRD and DSC consistent with |
| | | | | | mainly amorphous material. |
| Indomethacin | E100 | EtOH:Ace | 10 | 1:1 | Discrete, mainly spheroidal, ~200-500 nm |
| | | | | | particles. XRD and DSC consistent with |
| | | | | | mainly amorphous material. |
| Indomethacin | HPMC- | MeOH:Ace | 10 | 1:1 | Irregular, rough-to-wrinkled, ovoid to short |
| | AS | | | | rod shaped particles, ~0.5 to 1.0 μm. XRD |
| | | | | | and DSC consistent with mainly amorphous |
| | | | | | material. |
| Itraconazole | K30 plus | EtOH:Ace with | 10 | 1:4:1 | Dimpled spheroidal 0.8-1.4 μm discrete |
| | Gelucire | dichloromethane | | (ITZ:K30:Gelucire) | particles with some very fine fibrillary |
| | 14/44 | | | | extensions and satellite particles. DSC |
| | | | | | consistent with partially amorphous material. |
| Celecoxib | None | EtOH:Ace | 10 | N/A | Irregular, thin, porous-appearing, flake-like |
| | | | | | particles, 0.5 to 2.0 μm. XRD and DSC |
| | | | | | consistent with mainly amorphous material. |
| Celocoxib | K30 | EtOH:Ace | 10 | 1:1 | Collapsed, spheroidal, near discoid, discrete |
| | | | | | ~0.7 to 0.9 μm particles. Some satellite |
| | | | | | particles present. XRD and DSC consistent |
| | | | | | with mainly amorphous material. |
| Celocoxib | E100 | EtOH:Ace | 10 | 1:1 | Discrete, spheroid, wrinkled surface, 0.8 |
| | | | | | to 1.0 μm particles. XRD and DSC |
| | | | | | consistent with mainly amorphous material. |
| Meloxicam | None | Dioxolane | 10 | N/A | Irregular, interconnected, rough-edged flakes |
| | | | | | ~1.0 to 1.5 μm in largest diameter with |
| | | | | | thickness ~100 nm. XRD and DSC |
| | | | | | consistent with crystalline material. |
| Meloxicam | K30 | Dioxolane | 5 | 1:1 | Irregular, interconnected, partially-rough- |
| | | | | | edged discoid 1.0 to 1.5 μm particles. XRD |
| | | | | | consistent with partially crystalline material. |
| Meloxicam | E100 | Dioxolane | 5 | 1:1 | Very thin discoid but discrete ~1.0 to 1.5 μm |
| | | | | | particles. XRD and DSC consistent with |
| | | | | | mainly amorphous material. |
| Meloxicam | HPMC- | Dioxolane | 5 | 1:1 | Collapsed, half-spheroidal ~1.5 to 3.0 μm |
| | AS | | | | thin-shelled particles with torn surfaces. |
| | | | | | XRD and DSC consistent with mainly |
| | | | | | crystalline material. |
| Diclofenac | None | EtOH:Ace | 10 | N/A | No discrete particles, rough-surfaced |
| | | | | | domains with possible crystalline features. |
| | | | | | XRD and DSC consistent with crystalline |
| | | | | | material. |
| Diclofenac | K30 | EtOH:Ace | 10 | 1:1 | Spheroidal, irregular, but discrete 0.3 to 0.8 μm |
| | | | | | particles. Some satellite particles. XRD |
| | | | | | and DSC consistent with mainly amorphous |
| | | | | | material. |
| Diclofenac | E100 | EtOH:Ace | 10 | 1:1 | Irregularly shaped but discrete small |
| | | | | | particles 0.2 to 0.4 μm. XRD and DSC are |
| | | | | | consistent with mainly amorphous material. |
| Diclofenac | HPMC- | EtOH:Ace | 10 | 1:1 | Flattened, irregularly-shaped but smooth- |
| | AS | | | | edged flakes, ~<1.0 μm in largest |
| | | | | | dimension. XRD and DSC consistent with |
| | | | | | partially amorphous material. |
| Aprepitant | None | EtOH:Ace | 10 | N/A | SEM showed 1.0-2.0 μm collapsed |
| | | 60:40 | | | spheroidal shapes, flattened edges ~200 nm |
| | | | | | in diameter. XRD and DSC consistent with |
| | | | | | mainly amorphous material. |
| Aprepitant | K30 | EtOH:Ace | 10 | 1:1 | Discrete discoid 1.0 to 2.0 μm particles. |
| | | 60:40 | | | Satellite particles present. XRD and DSC |
| | | | | | consistent with mainly amorphous material. |
| Aprepitant | E100 | EtOH:Ace | 10 | 1:1 | Collapsed, discrete but clumped spheroid 0.5 |
| | | | | | to 2.5 μm particles. A few satellite particles |
| | | | | | present. XRD and DSC consistent with |
| | | | | | mainly amorphous material. |
| Aprepitant | S100 | EtOH:Ace | 10 | 1:1 | Discrete, collapsed and wrinkled spheroidal |
| | | | | | 0.5 to 1.5 μm particles. XRD consistent with |
| | | | | | mainly amorphous material. DSC not tested. |
| Aprepitant | L100-55 | EtOH:Ace | 10 | 1:1 | Discrete, collapsed 1.0 to 2.0 μm particles |
| | | | | | interspersed with many smaller 0.05 μm |
| | | | | | particles with similar appearance. XRD |
| | | | | | consistent with mainly amorphous material. |
| | | | | | DSC not tested. |
| Aprepitant | HPMC- | MeOH:Ace | 10 | 1:1 | Flattened, discoid, non-discrete and partially |
| | AS | | | | fused 0.5 to 2.0 μm particles. XRD and DSC |
| | | | | | consistent with mainly amorphous material. |
| Aprepitant | SoluPlus | EtOH:Ace | 10 | 1:1 | Discrete, discoid 1.5 to 2.5 μm particles. |
| | | | | | DSC is consistent with mainly amorphous |
| | | | | | material. XRD not tested. |

HPMC-AS = hydroxypropylmethylcellulose-acetate-succinate
MeOH = methanol
Gelucire 14/44 = lauroyl polyoxylglycerides (Gattefossé Corporation, Paramus, New Jersey)
S100 = Eudragit S100 (Evonik, Germany)

### Control C1 and Examples C1-C4

Control C1 and Examples C1-C4 were prepared as described above in Example B with the exception that the compound was aprepitant and the type of polymer and the ratio of aprepitant to polymer were as follows: Control C1 is 100% reference listed drug, Example C1 E100 1:2, Example C2 E100 1:1.5, Example C3 HPMC-AS 1:1.5, and Example C4 HPMC-AS 1:2.

Control C1 and Examples C1-C4 were tested according to the Dissolution in Biorelevant Medium Using Non-Sink Condition Test Method with the exception that the 100 mL dissolution vessels were used instead of 150 mL Erlenmeyer flasks, sampling was stopped after 120 minutes, and the number of samples for each example or control was six.

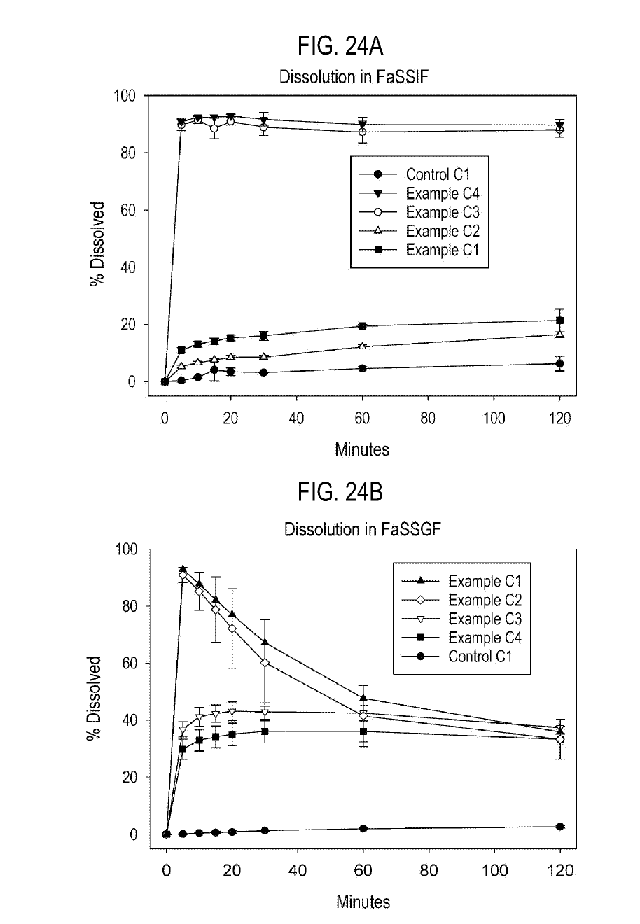

The percent dissolved aprepitant in FaSSIF versus time for each of Control C1 and Examples C2, C1, C3, and C4, respectively, is plotted (from bottom to top) in FIG. 24A.

The percent dissolved aprepitant in FaSSGF versus time for each of Control C1 and Examples C1-C4 is plotted in FIG. 24B as, from bottom to top at 20 minutes: Control C1, Example C4, Example C3, Example C2, and Example C1, respectively.

Other embodiments are within the claims. All references referred to herein are incorporated in their entirety to the extent they do not conflict with statements herein.

What is claimed is:

1. A method of converting a poorly water soluble crystalline compound into an amorphous compound, the method comprising:
dissolving the crystalline compound and a polymer in a solvent to form a solution, the polymer being present in the solution in an amount such that, after electrospraying the solution, the compound is in an amorphous form;
electrospraying the solution using an electrospray device, the electrospraying forming amorphous nanoparticles; and
collecting the nanoparticles on a substrate; and
removing the nanoparticles from the substrate in the form of a dry powder,
the nanoparticles comprising the compound in an amorphous form.

2. The method of claim 1 further comprising:
dissolving the compound in a first solvent to form a first solution;
dissolving the polymer in a second solvent to form a second solution; and
combining the first solution and the second solution to form a third solution,
the electrospraying comprising electrospraying the third solution using the electrospray device.

3. The method of claim 1, wherein the nanoparticles comprise amorphous nanoparticles.

4. The method of claim 1, wherein the nanoparticles are amorphous and remain amorphous when stored in a sealed container for at least one week at room temperature and ambient humidity.

5. The method of claim 1, wherein the nanoparticles are amorphous and remain amorphous when stored in a sealed container for at least two weeks at room temperature and ambient humidity.

6. The method of claim 1, wherein the nanoparticles exhibit a particle size in FaSSIF having a pH of at least 6.5 that is smaller than the particle size of the crystalline compound electrosprayed in the absence of the polymer in FaSSIF having a pH of at least 6.5.

7. The method of claim 1, wherein the nanoparticles achieve supersaturation of the amorphous compound in FaSSIF having a pH of at least 6.5.

8. The method of claim 1, wherein the nanoparticles achieve supersaturation of the amorphous compound in FaSSIF having a pH of at least 6.5 for a period of at least 60 minutes.

9. The method of claim 1, wherein the nanoparticles achieve supersaturation of the amorphous compound in FaSSIF having a pH of at least 6.5 for a period of at least 60 minutes, after exposure to FaSSGF having a pH of 1.6 for 30 minutes.

10. The method of claim 1, wherein the nanoparticles achieve supersaturation of the amorphous compound in FaSSIF having a pH of at least 6.5 for a period of at least 120 minutes.

11. The method of claim 1, wherein the nanoparticles achieve supersaturation of the amorphous compound in FaSSGF having a pH of no greater than 1.6.

12. The method of claim 1, wherein the nanoparticles exhibit a smaller particle size in FaSSGF having a pH of no greater than 1.6 relative to the size of particles of the compound electrosprayed in the absence of polymer in FaSSGF having a pH of no greater than 1.6.

13. The method of claim 1, wherein the nanoparticles achieve a greater solubility of the amorphous compound in FaSSIF having a pH of at least 6.5 than in FaSSGF having a pH no greater than 1.6.

14. The method of claim 1, wherein the nanoparticles achieve a greater solubility of the amorphous compound in FaSSGF having a pH no greater than 1.6 than in FaSSIF having a pH of at least 6.5.

15. The method of claim 1, wherein the polymer comprises an amorphous polymer.

16. The method of claim 1, wherein the polymer comprises an amorphous, water insoluble polymer.

17. The method of claim 1, wherein the polymer comprises at least one of a cationic polymer and an anionic polymer.

18. The method of claim 1, wherein the compound is anionic and the polymer comprises a cationic polymer.

19. The method of claim 1, wherein the crystalline compound comprises a crystalline drug.

20. The method of claim 1, wherein the crystalline compound comprises at least one of an antifungal drug, a non-steroidal anti-inflammatory drug, a corticosteroid, and a substance P antagonist.

21. A particulate comprising:
amorphous nanoparticles comprising
an amorphous compound, and
an amorphous polymer,
the nanoparticles having been formed by electrospraying a solution from an electrospray device, the solution comprising the polymer and solvent and having been derived from the compound in its crystalline form.

22. The particulate of claim 21, wherein the particulate is a dry powder.

23. The particulate of claim 21, wherein the nanoparticles comprise spheroidal nanoparticles with surface dimpling, discoid nanoparticles, teardrop-spheroidal nanoparticles, wrinkled spheroidal nanoparticles, porous spheroidal nanoparticles, pitted spheroidal nanoparticles, or a combination thereof.

24. A method of increasing the solubility of a poorly water soluble crystalline compound in a biorelevant fluid having a pH of at least 6.5, the method comprising:
dissolving the compound and a polymer in a solvent to form a solution, the polymer being present in the solution in an amount such that, after electrospraying the solution, the compound is in an amorphous form;
electrospraying the solution using an electrospray device, the electrospraying forming nanoparticles;
collecting the nanoparticles on a substrate, and
removing the nanoparticles from the substrate in the form of a dry powder, the nanoparticles comprising the compound in an amorphous form.

25. The particulate of claim 21, wherein the amorphous compound comprises amorphous aprepitant, and the amorphous polymer comprises hydroxypropylmethylcellulose acetate succinate.

26. The particulate of claim 25, wherein at least 80% of the amorphous aprepitant in the nanoparticles dissolves in FaSSIF in less than 10 minutes.

27. The particulate of claim 25, wherein the amorphous aprepitant and the hydroxypropylmethylcellulose acetate succinate are present in the nanoparticles in an aprepitant to hydroxypropylmethylcellulose acetate succinate weight to weight ratio of from 1:1 to 1:2.
